(19) United States
(12) Patent Application Publication
(10) Pub. No.: US 2011/0177098 A1
(43) Pub. Date: Jul. 21, 2011

(54) TM4SF4 AND MODULATORS THEREOF AND METHODS FOR THEIR USE

(76) Inventors: Lori Sussel, New York, NY (US); Keith Robert Anderson, Berkeley, CA (US)

(21) Appl. No.: 13/054,448

(22) PCT Filed: Jul. 15, 2009
(86) PCT No.: PCT/US2009/004121

§ 371 Date: Mar. 10, 2011

Related U.S. Application Data
(60) Provisional application No. 61/135,009, filed on Jul. 15, 2008.

(51) Int. Cl. A61K 39/395; C12N 5/0781; A61K 38/02; A61K 31/7088; A61K 31/715; A61K 31/713; A61K 35/39; A61P 3/10

(52) U.S. Cl. 424/173.1; 435/375; 514/1.1; 514/44 R; 514/54; 514/44 A; 424/93.71

(57) ABSTRACT

The present invention relates to methods for modulating a β-cell population using a TM4SF4 modulator or a modulator of a TM4SF4 homolog. More particularly, the invention relates, inter alia, to methods and compositions for generating, expanding, and maintaining a β-cell population and treatment of diseases associated with the loss of β-cells using a TM4SF4 modulator or a modulator of a TM4SF4 homolog.

### CROSS-REFERENCE TO RELATED APPLICATIONS

The present application claims benefit to U.S. provisional application Ser. No. 61/135,009 filed 15 Jul. 2008, the entire contents of which is incorporated by reference.

### FEDERALLY SPONSORED RESEARCH OR DEVELOPMENT

This invention was made with government support under UO1-DK072504 and VUMC 33065-R (subcontract issued from grant number U19 DK0724473) awarded by The National Institute of Diabetes and Digestive and Kidney Diseases (NIDDK), which is a part of the National Institutes of Health. The government has certain rights in the invention.

### FIELD OF THE INVENTION

The present invention relates, inter alia, to methods and compositions for modulating a β-cell population, particularly insulin-producing β-cell populations, using TM4SF4 modulators.

### BACKGROUND OF THE INVENTION

A major goal in modern diabetes research is to develop a source of human islets ex vivo that may be used to replenish β-cells destroyed in Type 1 diabetic patients. Such efforts involve working towards inducing endocrine stem or progenitor cells to differentiate into functional β-cells. Insight into the central role that pancreas-enriched transcription factors play in β-cell formation has been obtained from gene ablation strategies in mice; however, the regulatory pathways controlled by these factors to direct islet cell differentiation are not well understood.

Although some success has been achieved in the production of β-like cells from embryonic stem cells, the failure to induce or maintain the transcriptional activity of key regulators leads to the formation of non-functional cells. One way to overcome this problem is to gain a better understanding of the precise transcriptional networks that regulate islet cell differentiation during embryonic development. Over the past fifteen years, major advances have been made in identifying many transcriptional regulators that are essential in the formation and maturation of islet β-cells. Combined gene deletion and targeted ectopic expression analyses have illustrated the spatial and temporal significance of these proteins and the complexity of the processes they mediate. Furthermore, the use of tissue and temporally regulated gene deletion and misexpression strategies has begun to elucidate the distinct stage(s) of β-cell formation that each of these factors are involved in.

To efficiently and effectively produce functional n-cells for therapeutic purposes, a thorough understanding of the regulatory networks normally initiated in vivo by critical β-cell regulators must be achieved. Nkx2.2 is a transcription factor that has been shown to play a critical role at distinct stages of islet development. The homeodomain transcription factor Nkx2.2 is essential for pancreatic islet development and islet cell type differentiation; Nkx2.2 null mutations lead to a misspecification of islet cell types. To understand the molecular role of Nkx2.2 in islet cell specification, it will be important to identify direct transcriptional targets that regulate this important developmental process.

In view of the foregoing, it would be advantageous to elucidate the signaling pathways for and molecular regulation of the generation, expansion and maintenance of β-cells. It would also be advantageous to use such insights to provide new methods and compositions for generating, expanding, and maintaining β-cells in vivo and ex vivo, which β-cells would be of the kind and quantity sufficient to treat a patient with a disease associated with the loss of β-cells, such as diabetes.

### SUMMARY OF THE INVENTION

Thus, one embodiment of the invention is a method for modulating a β-cellpopulation. This method comprises contacting an islet progenitor cell population with an amount of a transmembrane 4 superfamily member 4 (TM4SF4) modulator or a modulator of a TM4SF4 homolog, which is sufficient to modulate the production of β-cell population.

Another embodiment of the invention is a pharmaceutical composition. This pharmaceutical composition comprises a pharmaceutically acceptable carrier and a TM4SF4 modulator or a modulator of a TM4SF4 homolog, which modulator is present in the composition in an amount sufficient to modulate production of a β-cell population when administered to a patient in need thereof.

Yet another embodiment of the invention is a method for expanding a β-cell population. This method comprises contacting an islet progenitor cell population comprising a β-cell with a TM4SF4 modulator or a modulator of a TM4SF4 homolog for a period of time sufficient to expand the number of β-cells in the population.

An additional embodiment of the invention is a method for treating a disease associated with a loss of β-cells. This method comprises administering to a patient in need thereof an expanded β-cell population obtained from the methods of this invention.

Another embodiment of the invention is a method for treating a disease associated with a loss of β-cells. This method comprises administering to a patient in need thereof a pharmaceutical composition according to the present invention.

A further embodiment of the invention is a method of increasing the production of β-cells from a cell population. This method comprises contacting the cell population with a TM4SF4 inhibitor or an inhibitor of a TM4SF4 homolog.

These and other aspects of the invention are further disclosed in the detailed description and examples which follow.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

The application contains at least one drawing executed in color. Copies of this patent and/or application publication with color drawing(s) will be provided by the Office upon request and payment of the necessary fee.

FIG. 1 is a schematic showing the expression of Nkx2.2 in various types of islet cells.

FIG. 2 shows that Nkx2.2 null mice (Nkx2.2 KO) have no 13 and few a cells. Cells expressing ghrelin are abundant in Nkx2.2 null mice. In the left panels, green indicates the location of amylase, red indicates the location of insulin, and blue indicates the location of glucagon. In the right panels, red indicates the location of ghrenlin.

FIG. 3 shows that the expression level of TM4SF4 is much higher in Nkx2.2 null mice (KO) than in the wild type (WT). FIG. 3A shows the results of a real time PCR analysis in embryonic day (e) 13.5 and e 18.5 mouse embryos. FIG. 3B shows an in situ analysis of TM4SF4 expression in e 17.5 mouse embryos. The magnification is 20×.

FIG. 4 shows in situ analysis of Nkx2.2 expression in 48 hour post fertilization (hpf) zebrafish. As shown, Nkx2.2 is expressed in the pancreas (for details, see panels B and C). Nkx2.2 expression is conserved between zebrafish and mice (data not shown).

FIG. 5 shows in situ analysis of TM4SF4 expression in zebrafish. In wild type (WT) zebrafish, TM4SF4 is expressed in the liver at approximately 46 hpf. In Nkx2.2 knockdown zebrafish (10 ng Nkx2.2MO), TM4SF4 is expressed in the liver and apparently in pancreas and intestine as well.

FIG. 6 shows analysis of insulin expression in zebrafish at 48 hpf. In TM4SF4 knockdown zebrafish (Tm4sf MO) insulin-expressing cells are increased in comparison to wild-type zebrafish (WT).

FIG. 7 shows a close-up analysis of insulin expression in zebrafish at 48 hpf after the removal of the yolk sac. Again, insulin-expressing cells are increased in TM4SF4 knockdown zebrafish (5 ng TM4SF4 splice MO) in comparison to wild type (WT) zebrafish. The magnification is as labeled.

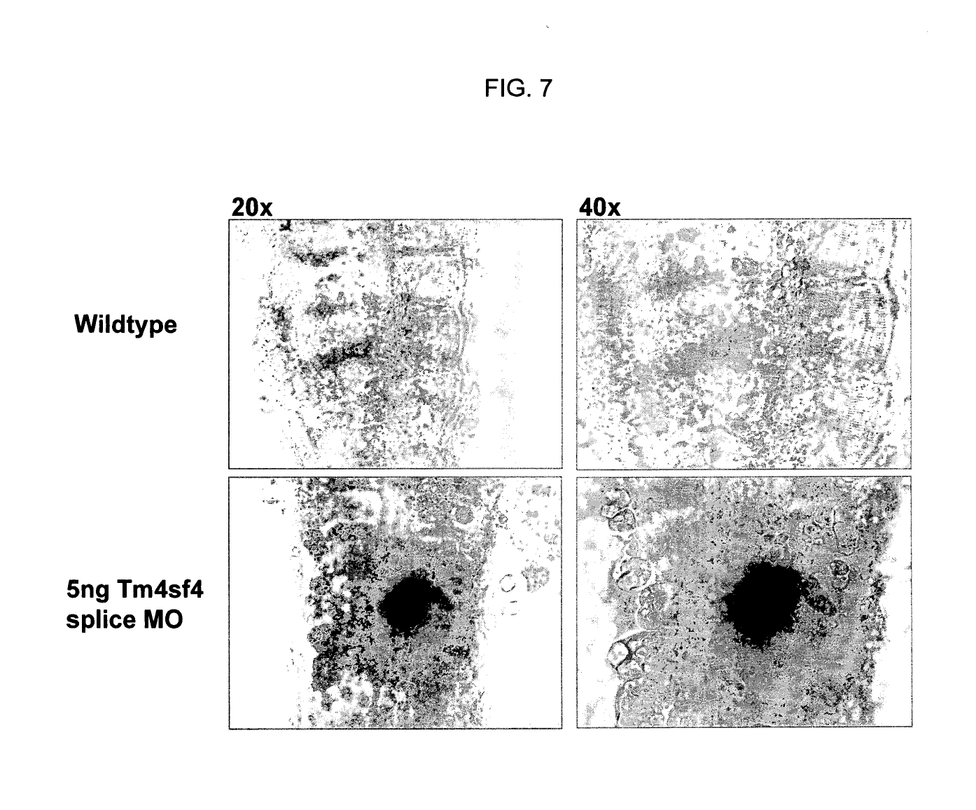

FIG. 8 shows an analysis of mouse TM4SF4 protein (SEQ ID NO: 29) domains based on protein modeling. Potential intracellular regions are shown in boxes with solid outlines, potential extracellular regions are shown in boxes with dashed outlines, and the potential N-linked glycosylation site is underlined.

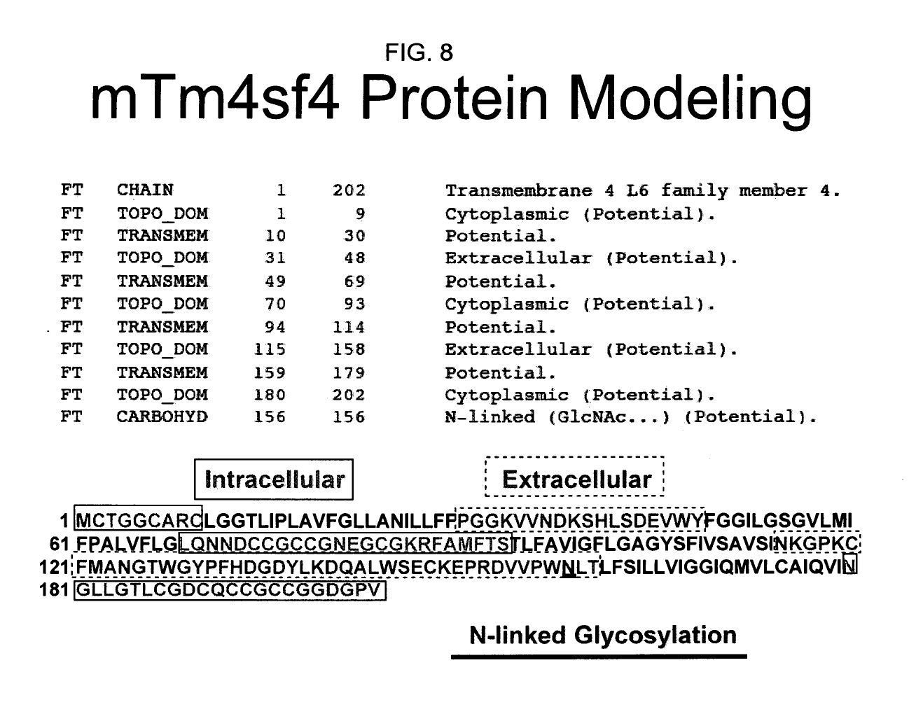

FIG. 9 shows quantitative real time PCR of the zebrafish hormones, insulin, glucagon, ghrelin, and somatostatin, in fish treated with Tm4sf4 morpholinos to knockdown Tm4sf4 or in wild type controls. The samples were normalized to β-actin. Each sample group contained 14 pooled embryos. The transcription factors Pdx1 and Nkx2.2a were also analyzed. Asterisk (*) indicates p<0.05, using a student's t-test.

FIG. 10 shows a schematic of the Tm4sf4 GFP knockout/knockin construct.

FIG. 11 shows staining of e15.5 pancreas (20×). Blue indicates the location of nuclei, red indicates the location of insulin, and green indicates the location of glucagon.

FIG. 12 shows the results of real time PCR in E18.5 Tm4sf4^{-/-} mouse pancreas. The amount of insulin and glucagon expression is increased in these knockout mice.

FIG. 13 shows a map of the pSP72:GFPLNL cassette.

### DETAILED DESCRIPTION OF THE INVENTION

One embodiment of the invention is a method for modulating a β-cell population. This method comprises contacting an islet progenitor cell population with an amount of a transmembrane 4 superfamily member 4 (TM4SF4) modulator or a modulator of a TM4SF4 homolog, which is sufficient to modulate the production of 13-cell population.

As used herein, a “β-cell population” means a group of cells that corresponds to the insulin-producing cells in, e.g., the pancreas. A β-cell population may be cultured in vitro. It may be derived from cells in the pancreas or from progenitor cells, such as stem cells and islet progenitor cells. In the present invention, “insulin producing cells” include cells that synthesize (i.e., transcribe the insulin gene, translate the proinsulin mRNA, and modify the proinsulin mRNA into the insulin protein), express (i.e., manifest the phenotypic trait carried by the insulin gene), or secrete (release insulin into the extracellular space) insulin in a constitutive or inducible manner. Examples of known insulin producing cells include β-cells, which are located in the pancreatic islets in vivo. In order to secrete insulin, an insulin producing cell also must express IDX-1.

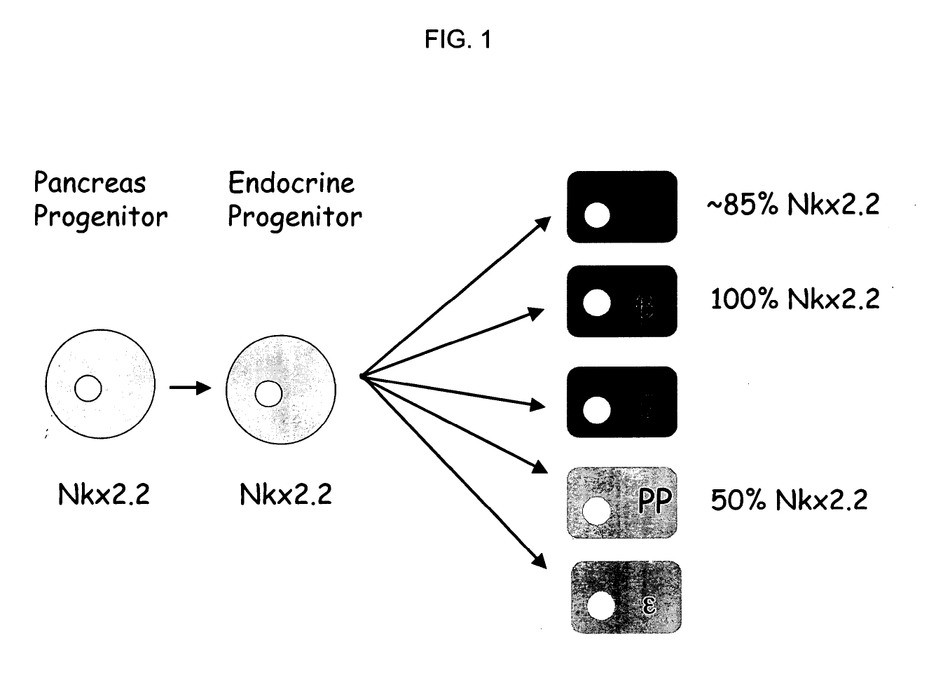

In the present invention, an “islet progenitor cell” is a precursor of all the cell types in the islet of Langerhans, including, e.g., α-, β-, δ-, PP, and ε-cells (see, e.g., FIG. 1), particularly, β-cells. Islet progenitor cells/cell populations are characterized by their expression of neurogenin 3 (Ngn-3).

As used herein, “modulate” “modulating,” and like terms mean to effect a change, directly or indirectly, in a target nucleic acid, polypeptide or the production of a cell population, such as the transcription of a target nucleic acid, the expression of a target polypeptide, the function of a polypeptide, the size of a cell population, the composition of a cell population, and/or the function of a cell population. For example, modulating the production of a β-cell population includes but is not limited to increasing the production of a β-cell population or increasing the formation, differentiation, expansion, maintenance, or the function (e.g., insulin-producing function) of a β-cell population, in, e.g., an islet progenitor cell population.

As used herein, “transmembrane 4 superfamily member 4” (TM4SF4) means the mouse TM4SF4 polypeptide sequence (SEQ ID NO: 29; cDNA shown in SEQ ID NO: 28). A “homolog” means a gene related to a second gene by descent from a common ancestral DNA sequence. Examples of TM4SF4 homologs include human intestinal and liver tetraspan membrane protein (il-TMP) (SEQ ID NO: 1; cDNA shown in SEQ ID NO: 2) and zebrafish TM4SF4 (SEQ ID NO:31; cDNA shown in SEQ ID NO: 30).

As used herein, a “modulator” means an agent that can alter the signal transduction pathway, e.g., the pathways of TM4SF4 or a TM4SF4 homolog, including but not limited to lowering or increasing the expression level of a protein, or reducing the activities of (including inhibiting the activities) or activating such a protein. Modulators may act by, for example, binding, phosphorylation, glycosylation, translocation of the protein; or they may act at the level of transcription or translation.

In one aspect of the embodiment, the TM4SF4 homolog is a human il-TMP. In another aspect of the embodiment, the TM4SF4 modulator inhibits TM4SF4 transcription, translation, or function.

In a further aspect of the embodiment, the TM4SF4 modulator is selected from the group consisting of nucleic acids, polypeptides, polysaccharides, small molecules, and combinations thereof.

Preferably, the modulator is selected from the group consisting of a fusion protein, an antibody, an antibody mimetic, a domain antibody, a targeted aptamer, an RNAi, an siRNA, an shRNA, an antisense sequence, and combinations thereof.

In the present invention, the term “nucleic acids” means large molecules composed of one or more chains of monomeric nucleotides, nucleotide-analogs, or combinations thereof. Nucleic acids may be single or double-stranded. Nucleotides refer to the building blocks of naturally occurring nucleic acids, such as deoxyribonucleic acid (DNA) and ribonucleic acid (RNA), and is comprised of a phosphate group, a sugar, and a purine or pyrimidine base. Nucleotide-analogs refer to chemically modified nucleotides. As used herein, nucleic acids include naturally occurring nucleic acids, as well as artificial nucleic acids, such as morpholinos, in which the bases are bound to morpholine rings instead of deoxyribose or ribose rings and linked through phosphorodiamidate groups instead of phosphates. Nucleic acids include RNAi, siRNA, shRNA and antisense sequences.

The term “RNA interference” (“RNAi”) refers to nucleic acids that are able to induce the RNA interference pathway. (Elbashir, S. M. et al. Nature 411:494-498 (2001); Caplen, N. J. et al. Proc. Natl. Acad. Sci. USA 98:9742-9747 (2001); Harborth, J. et al. J Cell Sci. 114:4557-4565 (2001).) RNAi may be delivered into the cell via vectors or via adenovirus.

The term small interfering RNA (“siRNA”) refers to small inhibitory RNA duplexes that induce the RNA interference pathway. These molecules may vary in length (generally 18-30 base pairs) and contain varying degrees of complementarity to their target mRNA in the antisense strand. Some, but not all, siRNA have unpaired overhanging bases on the 5′ or 3′ end of the sense strand and/or the antisense strand. The term “siRNA” includes duplexes of two separate strands. As used herein, siRNA molecules are not limited to RNA molecules but further encompass nucleic acids with one or more chemically modified nucleotides, such as morpholinos. siRNA gene-targeting may be carried out by transient siRNA transfer into cells (achieved by such classic methods as liposome-mediated transfection, electroporation, or microinjection).

An “antisense sequence,” as used herein includes antisense or sense oligonucleotides comprising a single-stranded nucleic acid sequence (either RNA or DNA) capable of binding to target mRNA (sense) or DNA (antisense) sequences. The ability to derive an antisense or a sense oligonucleotide, based upon a cDNA sequence encoding a given protein is described in, for example, Stein and Cohen, Cancer Res. 48:2659, (1988) and van der Krol et al., BioTechniques 6:958, (1988).

Antisense molecules may be modified or unmodified RNA, DNA, or mixed polymer oligonucleotides. These molecules function by specifically binding to matching sequences resulting in inhibition of peptide synthesis (Wu-Pong, November 1994, BioPharm, 20-33) either by steric blocking or by activating an RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190). In addition, binding of single stranded DNA to RNA may result in nuclease-mediated degradation of the heteroduplex (Wu-Pong, supra). Backbone modified DNA chemistry, which may to act as substrates for RNase H include, e.g., phosphorothioates, phosphorodithioates, borontrifluoridates, and 2′-arabino and 2′-fluoro arabino-containing oligonucleotides.

Antisense sequences may be introduced into a cell containing the target nucleotide sequence by formation of a conjugate with a ligand binding molecule, as described, e.g., in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or to block entry of the sense or antisense oligonucleotide or its conjugated version into the cell. Alternatively, a sense or an antisense oligonucleotide may be introduced into a cell containing the target nucleic acid sequence by formation of an oligonucleotide-lipid complex, as described, e.g., in WO 90/10448.

As used herein, a “small hairpin RNA” or “short hairpin RNA” (shRNA) is a sequence of RNA that makes a tight hairpin turn that can be used to silence gene expression via RNA interference. shRNA may be introduced into cells via a vector. As used herein, shRNA molecules are not limited to RNA molecules but further encompass nucleic acids with one or more chemically modified nucleotides.

In the present invention, the terms “peptide,” “polypeptide” and “protein” are used interchangeably. They refer to a linked sequence of two or more amino acids, which may be natural, synthetic, or a modification or combination of natural and synthetic. Such terms include fusion proteins, peptidomimetic, antibodies, antibody mimetics, domain antibodies, and targeted proteases, which are capable of, e.g., substrate-targeted inhibition of post-translational modification such as disclosed in, e.g., U.S. Patent Application Publication No. 20060275823. The term also includes vaccines containing a peptide or peptide fragment intended to raise antibodies against the polypeptide or a fragment thereof.

As used herein, a “fusion protein” means a polypeptide in which two or more proteins, whether wild-type, mutated, or truncated, are joined together. The joining may occur via, for example, molecular genetic techniques, wherein the polynucleotide sequences of the proteins are fused by polymerase chain reaction or by restriction sites.

In general, a polypeptide mimetic (“peptidomimetic”) is a molecule that mimics the biological activity of a polypeptide, but that is not peptidic in chemical nature. While, in certain embodiments, a peptidomimetic is a molecule that contains no peptide bonds (that is, amide bonds between amino acids), the term peptidomimetic may include molecules that are not completely peptidic in character, such as pseudo-peptides, semi-peptides, and peptoids.

“Antibody” as used herein includes an antibody of classes IgG, IgM, IgA, IgD, or IgE, or fragments or derivatives thereof, including Fab, F(ab′)2, Fd, and single chain antibodies, diabodies, bispecific antibodies, and bifunctional antibodies. The antibody may be a monoclonal antibody, polyclonal antibody, affinity purified antibody, or mixtures thereof, which exhibits sufficient binding specificity to a desired epitope or a sequence derived therefrom. The antibody may also be a chimeric antibody. The antibody may be derivatized by the attachment of one or more chemical, peptide, or polypeptide moieties known in the art. The antibody may be conjugated with a chemical moiety. The antibody may be a human or humanized antibody. These and other antibodies are disclosed in, e.g., U.S. Published Patent Application No. 20070065447.

Other antibody-like molecules are also within the scope of the present invention. Such antibody-like molecules include, e.g., receptor traps (such as entanercept), antibody mimetics (such as adnectins, fibronectin based “addressable” therapeutic binding molecules from, e.g., Compound Therapeutics, Inc.), domain antibodies (the smallest functional fragment of a naturally occurring single-domain antibody (such as, e.g., nanobodies; see, e.g., Cortez-Retamozo et al., Cancer Res. 2004 Apr. 15; 64(8):2853-7)).

Suitable antibody mimetics generally can be used as surrogates for the antibodies and antibody fragments described herein. Such antibody mimetics may be associated with advantageous properties (e.g., they may be water soluble, resistant to proteolysis, and/or be nonimmunogenic). For example, peptides comprising a synthetic beta-loop structure that mimics the second complementarity-determining region (CDR) of monoclonal antibodies have been proposed and generated. See, e.g., Saragovi et al., Science. Aug. 16, 1991; 253(5021):792-5. Peptide antibody mimetics also have been generated by use of peptide mapping to determine “active” antigen recognition residues, molecular modeling, and a molecular dynamics trajectory analysis, so as to design a peptide mimic containing antigen contact residues from multiple CDRs. See, e.g., Cassett et al., Biochem Biophys Res Commun. Jul. 18, 2003; 307(1):198-205. Additional discussion of related principles, methods, etc., that may be applicable in the context of this invention are provided in, e.g., Fassina, Immunomethods. October 1994; 5(2):121-9.

As used herein, a “polysaccharide” means a polymer composed of more than one monosaccharide unit. The monosaccharide units may be of the same type, or of different types.

In the present invention, the term “small molecule” includes any chemical or other moiety, other than polypeptides, nucleic acids, and polysaccharides, that can act to affect biological processes. Small molecules may include any number of therapeutic agents presently known and used, or that can be synthesized in a library of such molecules for the purpose of screening for biological function(s). Small molecules are distinguished from macromolecules by size. The small molecules of the present invention usually have a molecular weight less than about 5,000 daltons (Da), preferably less than about 2,500 Da, more preferably less than 1,000 Da, most preferably less than about 500 Da.

Small molecules include without limitation organic compounds and conjugates thereof. As used herein, the term “organic compound” refers to any carbon-based compound other than macromolecules such as nucleic acids and polypeptides. In addition to carbon, organic compounds may contain calcium, chlorine, fluorine, copper, hydrogen, iron, potassium, nitrogen, oxygen, sulfur and other elements. An organic compound may be in an aromatic or aliphatic form. Non-limiting examples of organic compounds include acetones, alcohols, anilines, carbohydrates, monosaccharides, amino acids, nucleosides, nucleotides, lipids, retinoids, steroids, proteoglycans, ketones, aldehydes, saturated, unsaturated and polyunsaturated fats, oils and waxes, alkenes, esters, ethers, thiols, sulfides, cyclic compounds, heterocyclic compounds, imidizoles, and phenols. An organic compound as used herein also includes nitrated organic compounds and halogenated (e.g., chlorinated) organic compounds.

Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

As used herein, a “targeted aptamer” means a molecule that binds to a specific molecular target. A targeted aptamer may be a peptide or a nucleic acid. Typically, a peptide aptamer consists of a variable peptide loop attached at both ends to a protein scaffold.

In another aspect of the embodiment, the modulator acts upstream of TM4SF4. In a further aspect, the modulator acts downstream of TM4SF4. As used herein, acting “upstream” of TM4SF4 means acting on a molecule that directly or indirectly affects TM4SF4, e.g., affecting the expression level of TM4SF4 or affecting the activities of TM4SF4. Thus, if a modulator acts upstream of TM4SF4, the modulator acts on another molecule e.g., Nkx2.2, which in turn, affects TM4SF4. Acting “downstream” of TM4SF4 means acting on a molecule, which TM4SF4 directly or indirectly affects. Thus, if a modulator acts downstream of TM4SF4, the modulator acts on a molecule that is below TM4SF4 in the pathway.

Another embodiment of the invention is a pharmaceutical composition. This pharmaceutical composition comprises a pharmaceutically acceptable carrier and a TM4SF4 modulator or a modulator of a TM4SF4 homolog, which modulator is present in the composition in an amount sufficient to modulate production of a β-cell population when administered to a patient in need thereof.

Pharmaceutically acceptable carriers are well known in the art (see, e.g., Remington's Pharmaceutical Sciences (Mack Publishing Co., Easton, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition comprising a modulator of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration may be determined using ordinary skill in the art.

Pharmaceutical compositions comprising a modulator of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions for rectal or vaginal administration may be presented as a suppository, which maybe prepared by mixing one or more active ingredient(s) with one or more suitable nonirritating carriers which are solid at room temperature, but liquid at body temperature and, therefore, will melt in the rectum or vaginal cavity and release the active compound. Pharmaceutical compositions which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active compound may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administrations comprise, e.g., one or more TM4SF4 modulators in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug containing, e.g., a TM4SF4 modulator of the present invention, it is desirable to slow its absorption from subcutaneous or intramuscular injection. This may be accomplished by the use of a liquid suspension of crystalline or amorphous material having poor water solubility.

The rate of absorption of the drug then depends upon its rate of dissolution which, in turn, may depend upon crystal size and crystalline form. Alternatively, delayed absorption of a parenterally-administered drug may be accomplished by dissolving or suspending the drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

In one aspect of this embodiment, the TM4SF4 homolog is a human il-TMP. In another aspect of the embodiment, the TM4SF4 modulator inhibits TM4SF4 transcription, translation, and/or function.

In a further aspect of this embodiment, the TM4SF4 modulator is selected from the group consisting of nucleic acids, polypeptides, polysaccharides, small molecules, and combinations thereof. Preferably, the modulator is selected from the group consisting of a fusion protein, an antibody, an antibody mimetic, a domain antibody, a targeted aptamer, an RNAi, an siRNA, an shRNA, an antisense sequence, and combinations thereof.

In an additional aspect of this embodiment, the modulator acts upstream or downstream of TM4SF4.

In a further aspect of this embodiment, the TM4SF4 modulator or the modulator of the TM4SF4 homolog increases production of the β-cell population.

An additional embodiment of the invention is a method for expanding a β-cell population. This method comprises contacting an islet progenitor cell population comprising a β-cell with a TM4SF4 modulator or a modulator of a TM4SF4 homolog for a period of time sufficient to expand the number of β-cells in the population.

In one aspect of this embodiment, the TM4SF4 homolog is a human il-TMP. In another aspect of this embodiment, the contacting step comprises administering the TM4SF4 modulator to a patient, such as a mammal, preferably a human.

In a further aspect of this embodiment, the contacting step comprises ex vivo administration of the TM4SF4 to the cell population. As used herein, “ex vivo” means removing cells or tissues from the body and culturing such cells or tissues. Preferably, the islet progenitor cell population is obtained from a pancreas, preferably a mammalian, more preferably a human, pancreas.

In a further aspect of this embodiment, the TM4SF4 modulator inhibits TM4SF4 transcription, translation and/or functions. The TM4SF4 modulator is selected from the group consisting of nucleic acids, polypeptides, polysaccharides, small molecules, and combinations thereof. Preferably, the modulator is selected from the group consisting of a fusion protein, an antibody, an antibody mimetic, a domain antibody, a targeted aptamer, an RNAi, an siRNA, an shRNA, an antisense sequence, and combinations thereof.

In an additional aspect of the embodiment, the modulator acts upstream or downstream of TM4SF4.

Another embodiment of the invention is a method for treating a disease associated with a loss of insulin producing cells, e.g., β-cells. This method comprises administering to a patient in need thereof an expanded β-cell population obtained from the methods of this invention.

In the present invention, a disease associated with the loss of β-cells means any condition characterized by a decreased level of insulin caused by, e.g., a decreased number of and/or function of insulin-producing cells, such as, e.g., β-cells. Representative diseases associated with the loss of β-cells include diabetes, such as type-I diabetes or type-II diabetes, particularly typed diabetes, as well as insulin resistance.

In one aspect of this embodiment, the expanded β-cell population is from an autologous source. In another aspect, the expanded β-cell population is from an heterologous source. As used herein, “autologous” means from the patient himself, and “heterologous” means from an individual other than the patient. Preferably, the patient is a mammal, such as, e.g., a human.

Another embodiment of the invention is a method for treating a disease associated with a loss of β-cells. This method comprises administering to a patient in need thereof any pharmaceutical composition according to the present invention.

In one aspect of this embodiment, the disease may be any condition characterized by a decrease in insulin caused by a decrease the numbers of or function of insulin producing cells, such as, e.g., β-cells. Such a disease may include diabetes, such as type-I diabetes or type-II diabetes, particularly type-I diabetes and insulin resistance.

A further embodiment of the invention is a method of increasing the production of β-cells from a cell population. This method comprises contacting the cell population with a TM4SF4 inhibitor or an inhibitor of a TM4SF4 homolog.

As used herein, an “inhibitor” means a molecule that can reduce the activities of the target or reduce the expression of the target (whether on a transcriptional or translational level). In one aspect of this embodiment, the TM4SF4 homolog is a human iI-TMP.

In another aspect of this embodiment, the cell population is selected from a mammalian, preferably human, embryonic stem cells or adult progenitor cells. As used herein, an “embryonic stem cell” means an undifferentiated cell from an embryo that has the potential to become a wide variety of specialized cell types, such as, e.g., a β-cell. An “adult progenitor cell” means an undifferentiated cell from a non-embryonic source that has the potential to become a variety of specialized cell types, such as, e.g., a β-cell.

In a further aspect of this embodiment, the cell population comprises islet progenitor cells.

The following examples are provided to further illustrate the methods and compositions of the present invention. These examples are illustrative only and are not intended to limit the scope of the invention in any way.

### EXAMPLES

### Example 1

### Immunofluorescence of Pancreas from Nkx2.2 Null Mice

Nkx2.2^{+/-} heterozygous mice were generated by homologous recombination (1). Briefly, to generate a Nkx2.2 knockout construct, three overlapping genomic clones were isolated from a phage library of mouse genomic sequences. The neomycin resistance gene was cloned into the genomic locus in place of the two Nkx2.2 coding exons. The knockout construct was introduced into ES cells by electroporation, selecting for Neo resistance. Cells containing the correct recombinant were identified by southern analysis. The Nkx2.2 KO ES cells were introduced into mice using standard technologies.

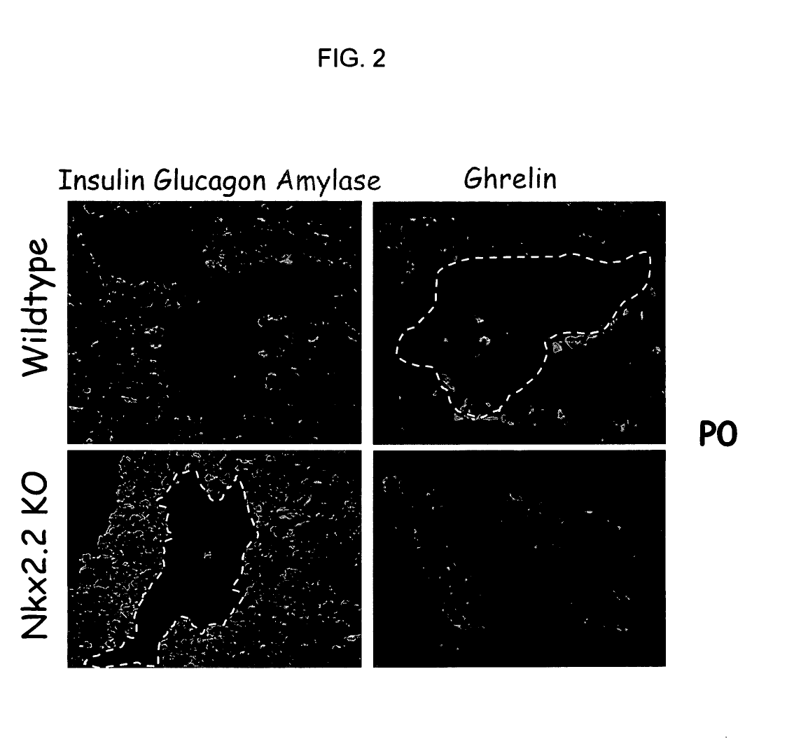

Nkx2.2^{+/-} heterozygous mice were maintained on a Swiss Black (Taconic) background. Genotyping of mice was performed by PCR analysis as described (1,9). P0 mice were harvested, fixed overnight in 4% paraformaldehyde, and cryoprotected in sucrose and OCT embedding material. Immunofluorescence was performed on frozen 10 μm sections. Antibodies used were mouse anti-insulin (1:1000, Sigma, St. Louis, Mo.), rabbit anti-amylase (1:1000; Sigma), guinea pig anti-glucagon (1:1000, Dako Denmark A/S, Glostrup, Denmark), and goat anti-ghrelin (1:200, Santa Cruz Biotechnologies, Inc., Santa Cruz, Calif.). Secondary antibodies (Jackson Immuno Research Laboratories, Inc., West Grove, Pa.) were against individual species, all raised in donkey, labeled with either Cy2 or Cy5, and used at 1:300. Confocal images were taken on a Zeiss META LSM 510. The results show that Nkx2.2 null mice (Nkx2.2 KO) have no β-cells and few a-cells (FIG. 2).

### Example 2

### Tissue Collection

Embryonic pancreas tissue (e 12.5 and e 13.5) was collected from either wild-type or Nkx2.2 knockout embryos. Tissue was dissected and stored in RNALater (Ambion, Houston, Tex.) until the genotypes were established. mRNA was extracted from the embryonic pancreas tissue using the RNeasy kit (Qiagen, Valencia, Calif.). The RNA was then shipped to the Penn Genomics Core (University of Pennsylvania, Philadelphia, Pa.) and processed for either the PancChip or the Agilent mouse chip.

### Example 3

### PancChip Analysis

The concentration of the nucleic acid samples was determined using the Nanoprop® ND-1000 UV-Vis Spectrophotometer. RNA samples were analyzed using an Agilent 2100 Bioanalyzer Lab-On-A-Chip Agilent 6000 Series II chip to determine the integrity of the samples.

### Experimental Design

In the current study, with one test and one control condition of interest, a direct comparison design was used. Each sample was labeled and hybridized as either Test (Cy5) vs. Control (Cy3) (M) or Control (Cy5) vs. Test (Cy3) (-M). After analysis, the M values were swapped so that Fold Changes were expressed as Test/Cont for each hybridization. The set up is shown in Table 1 below.

TABLE 1

| | | Cy-5 | Cy-3 | | |
|---|---|---|---|---|---|
| Hybridization | Barcode | (Red) | (Green) | Die Swap | comment |
| 1 | 13687835 | WT4 | KO4 | −M | |
| 2 | 13687836 | WT5 | KO5 | −M | |
| 3 | 13687870 | WT1 | KO1 | −M | |
| 4 | 13687871 | KO2 | WT2 | M | Hyb |
| | | | | | Rejected |
| 5 | 13687872 | KO3 | WT3 | M | |

### Labeling and Hybridization

Approximately 200 ng of total RNA was amplified using the MessageAmp™ II aRNA Amplification Kit (Ambion, Houston, Tex.). After amplification, 2.5 μg of amplified RNA (aRNA) was indirectly labeled using amino-allyl dUTP and anchored oligo(d)T prime reverse transcription. The cDNA was purified using the MinElute PCR Purification Kit (Qiagen, Valencia, Calif.), eluted in coupling buffer (0.1 M Sodium Bicarbonate, pH 9) and coupled with the appropriate Cy3 or Cy5 fluorescent label (Cy™Dye, Amersham Pharmacia Biotech Ltd, NJ), combined, and purified using the MinElute PCR Purification Kit (Qiagen, Valencia, Calif.).

After purification, 2.5 μg of Mouse Cot1 DNA (Invitrogen Life Technologies, Carlsbad, Calif.) and 2.5 μg Oligo-dT was added to each sample and denatured at 95° C. for 5 min. The samples were then cooled to 42° C. and an equal volume of 2× hybridization buffer (50% formamide, 10×SSC, and 0.2% SDS) was added, mixed, and applied to the array.

### Arrays

The Mouse PancChip 6 (The BCBC Functional Genomics Core, Nashville, Tenn.) was released in April 2005 and contains 13,059 mouse cDNAs chosen for their expression in various stages of pancreatic development, many of which are not found on commercially available arrays. This version of the PancChip represents a significant upgrade to the PancChip 5, with the addition of over 1000 full-length clones of particular importance to the field of pancreatic development and pathways relating to glucose homeostasis. Greater than 90% of the elements on the PancChip are also expressed in liver and colon.

### Scanning and Image Analysis

Microarray slides were hybridized overnight, then washed and scanned with an Agilent G2565BA Microarray Scanner. Images were analyzed with GenePix 5.0 software (Axon Instruments). Median foreground intensities were obtained for each spot and imported into the mathematical software package “R”, which was used for all data input, diagnostic plots, normalization and quality checking steps of the analysis process using scripts developed by Penn Genomics Core. The ratio of expression for each element on the array was calculated in terms of M (log₂(Red/Green)) and A ((log₂(Red)+log₂(Green))/2). The dataset was filtered to remove positive control elements (Cy3 anchors and SpotReport elements) and any elements that had been manually flagged as bad. The M values were then normalized by the print tip loess method using the “marray” microarray processing package in “R”. Statistical analysis was performed in “R” using both the LIMMA and SAM packages.

### Data Analysis

Microarrays measure the expression of thousands of genes to identify changes in expression between different biological states. As such, methods are needed to determine the significance of these changes while accounting for the enormous number of genes tested. The analysis is performed with 2-channel arrays, with condition 0 being hybridized to one channel and condition 1 hybridized to the other. Because the channels are not separated, but instead ratios are used, this requires an analysis which is somewhat different from the 1-channel (Affymetrix) or reference design experiments.

The M-value is the log₂-fold-change between two conditions. The A-value (A) is the mean log-expression level for that gene across both channels. M values are typically calculated as Log₂ (test/control), as such a positive M value (or fold change) indicates a gene that was upregulated in the test, while a negative M (or fold change) indicates a gene that was downregulated in the test. This is the simplest statistic used in microarray analysis whereby the mean of the M values is calculated for all replicates. The estimate of the fold change is calculated from this number. Typically, genes differentially expressed with a fold change <1.5 were not considered. However, for some genes a highly significant fold change that is <1.5 may have a significant biological impact. The Fold Change calculated from the normalized M value should always be used with an accompanying statistical test.

One significant challenge inherent to high-throughput analysis of large scale changes in gene expression is the development of statistical methods that will maximize both the sensitivity and specificity of detection of differentially expressed genes. The “SAM” statistical analysis package (Version 3.1) is a tool for analyzing microarray gene expression data useful for identifying differentially expressed genes between two conditions (2). SAM uses the False Discovery Rate (FDR) and q-value method (3). In the present invention, a one-class unpaired analysis with a FDR of 10% was used to maximize sensitivity without significantly impacting accuracy. Of the different statistical approaches to analysis of array data, this approach is considered to be the most robust.

The Log Odds Ratio method (lods ranking) uses a parametric empirical Bayes approach (4). The “B-statistic” is calculated estimating the posterior log odds that each gene is differentially expressed. This “lods” ratio is equivalent to a penalized t statistic: t=(mean M)/[(a+s²)/n]^{1/2} where a is the penalty estimated from the mean and the standard deviation of the sample variances s². Simulated data has shown this statistic to be superior to both t statistic and mean M. In essence, the LOD score is used to produce a ranked list (from the most significant to the least), and it is up to the investigator to decide how many of the top genes they want to follow up on. If one is willing to follow up on 10 genes, one would choose the top 10 genes to follow up on; if one can follow up on 100 genes, one would choose the top 100 and so on.

Adjusted p-value is another statistic measure. The moderated t-statistic (t) is the ratio of the M-value to its standard error. This has the same interpretation as an ordinary t-statistic except that the standard errors have been moderated across genes, i.e., shrunk towards a common value (5). This has the effect of borrowing information from the ensemble of genes to aid with inference about each individual gene. The p-value (p-value) is obtained from the distribution of the moderated t-statistic. Finally these p-values are adjusted for multiple testing using the Benjamini and Hochberg's (1995) step-up method for controlling the false discovery rate. This statistic should not be confused with the Students t test p-value.

### Results

Four of the five hybridizations worked well, and thus, an analysis using four biological replicates was performed. Table 2 below shows how many genes were called by SAM with increasing FDR (decreasing confidence), from 0% to 100%.

TABLE 2

| FDR | SAM Delta | Num Called |
|---|---|---|
| 0 | 1.472 | 111 |
| 0.5 | 1.468 | 112 |
| 1 | 1.054 | 154 |
| 2 | 0.727 | 216 |
| 5 | 0.514 | 288 |
| 10 | 0.35 | 416 |
| 15 | 0.269 | 511 |
| 20 | 0.222 | 629 |
| 25 | 0.187 | 910 |
| 30 | 0.181 | 1786 |
| 40 | 0.168 | 3540 |
| 50 | 0.156 | 5294 |
| 75 | 0 | 11251 |
| 100 | 0 | 11251 |

Tissue from Nkx2.2 wt and that from Nkx2.2 mutant mice were compared. Two sets of experiments (n=5 samples each experiment) at embryonic day (e) 13.5 and 1 set of experiments at e 12.5 were performed. All showed upregulation of TM4SF4 in Nkx2.2 mutant pancreas tissue. TM4SF4 was upregulated 3.32 fold (false discovery rate (FDR)=0) in e 12.5 Nkx 2.2 mutant mice. In mutant e 13.5 mice, TM4SF4 was upregulated 4.52 fold (FDR=0).

### Example 4

### Agilent Chip Analysis

The concentration of the nucleic acid samples was determined using the Nanoprop® ND-1000 UV-Vis Spectrophotometer. RNA samples were analyzed using an Agilent 2100 Bioanalyzer Lab-On-A-Chip Agilent 6000 Series II chip to determine the integrity of the samples. The RNA was of high quality and all samples passed QC cutoff.

### Experimental Design

Agilent's Dual-Mode Gene Expression Analysis Platform was used. In the present invention, with multiple conditions of interest, a single-color design was used. Each sample was labeled and hybridized as either Condition N (Cy3).

### Labeling and Hybridization

25 ng of total RNA was amplified using the Ovation™ RNA Amplification System V2 (Nugen Inc, CA). This system uses the rapid and sensitive Ribo-SPIA™ RNA amplification process, which involves a series of enzymatic reactions resulting in linear amplification of exceedingly small amounts of RNA for use in array analysis. Unlike exponential RNA amplification methods, such as NASBA and RT-PCR, Ovation amplification maintains representation of the starting mRNA population. The amplification process resulted in a yield of 6-10 μg of amplified cDNA, which was purified using the QIAquick PCR Purification Kit (Qiagen, Valencia, Calif.) and then eluted in coupling buffer (0.1 M Sodium Bicarbonate, pH 9). 2 μg of each amplified cDNA was labeled using the BioPrime® Array CGH Genomic Labeling System (Invitrogen Life Technologies, CA) as per the manufacturer's instructions. Briefly, 2 μg of cDNA was mixed with random primers and denatured at 95° C. for 5 min, then cooled briefly on ice. Next, the appropriate cyanine dUTP fluorescent nucleotide (GE, Piscataway, N.J.) was added, along with the nucleotide mix and Exo Klenow fragment. This was gently mixed and incubated at 37° C. for 2 hours. The Cy3 and Cy5 labeled samples were purified using the MinElute PCR Purification Kit (Qiagen, Valencia, Calif.), and the efficiency of dye incorporation and yield was determined using the Nanoprop® ND-1000 UV-Vis Spectrophotometer. After purification, 1.65 μg of cDNA was hybridized to the array for 17 hours at 65° C.

### Arrays

The Whole Mouse Genome Oligo Microarray G4122A is a broad view that represents all known genes and transcripts in the entire mouse genome. This 4 by 44K array is spotted with Agilent-designed 60-mer oligonucleotides, representing 21,609 known genes represented by 33,661 transcripts.

### Scanning and Image Analysis

Microarray slides were hybridized overnight, then washed and scanned with the Agilent G2565BA Microarray Scanner. Images were analyzed with Feature Extraction 9.5 (Agilent Technologies, CA). Mean foreground intensities were obtained for each spot and imported into the mathematical software package “R”, which is used for all data input, diagnostic plots, normalization and quality checking steps of the analysis process using scripts developed by Penn Functional Genomics Core. Briefly, the Cy3 (green) intensities were not background corrected (this has been shown to only introduce noise), and corrected for the scanner offset (40 was subtracted for each intensity). The dataset was filtered to remove positive control elements and any elements that had been flagged as bad. Using the negative controls on the arrays, the background threshold was determined and all values less than this value were set to the threshold value. Finally, the data was normalized using the Quantile Normalization package in “R” (6). This is a significantly more robust approach than simply normalizing to the median value of each array. Complete statistical analysis was then performed in “R” using both the LIMMA and SAM packages.

### Data Analysis

SAM analysis and adjusted p-value analysis are as described in Example 3 above.

Hierarchical clustering was performed on the samples (arrays) using the “R” package “pvclust” (7). This package calculates p-values for hierarchical clustering via multiscale bootstrap resampling. Hierarchical clustering is done for given data and p-values are computed for each of the clusters. It provides AU (approximately unbiased) p-values as well as BP (bootstrap probability) values computed via multiscale bootstrap resampling. One can consider that clusters (edges) with high AU values (e.g. 95%) are strongly supported by the data. Rectangles highlight those clusters with a highly significant P value (0.05), significant clusters within these highlighted clusters are not highlighted.

### Results

Nkx2.2. wt versus Nkx2.2 mutant tissue at e 12.5 were compared. TM4SF4 was upregulated 5-fold in Nkx2.2 mutants.

### Example 5

### Real Time PCR Analysis in Nkx2.2 Mutant Mice

Real time PCR analysis of TM4SF4 in wild type vs Nkx2.2 mutant pancreas at all ages of gestation was also carried out.

TM4SF4 AOD mix (Applied Biosystems, Pleasanton, Calif., catalog number Mm00523755_m1) was mixed with Taqman Buffer (Eurogentech, Seraing, Belgium) and appropriate amounts of DNA. For the standard curve setup, e 15.5 KO pancreas was used to make five 1:10 serial dilutions of the cDNA. For the experimental sample setup, cDNA made from 1 μg of RNA was used to determine concentration, and 40 ng/μl working stocks were made. A total of 200 ng of cDNA was added per reaction.

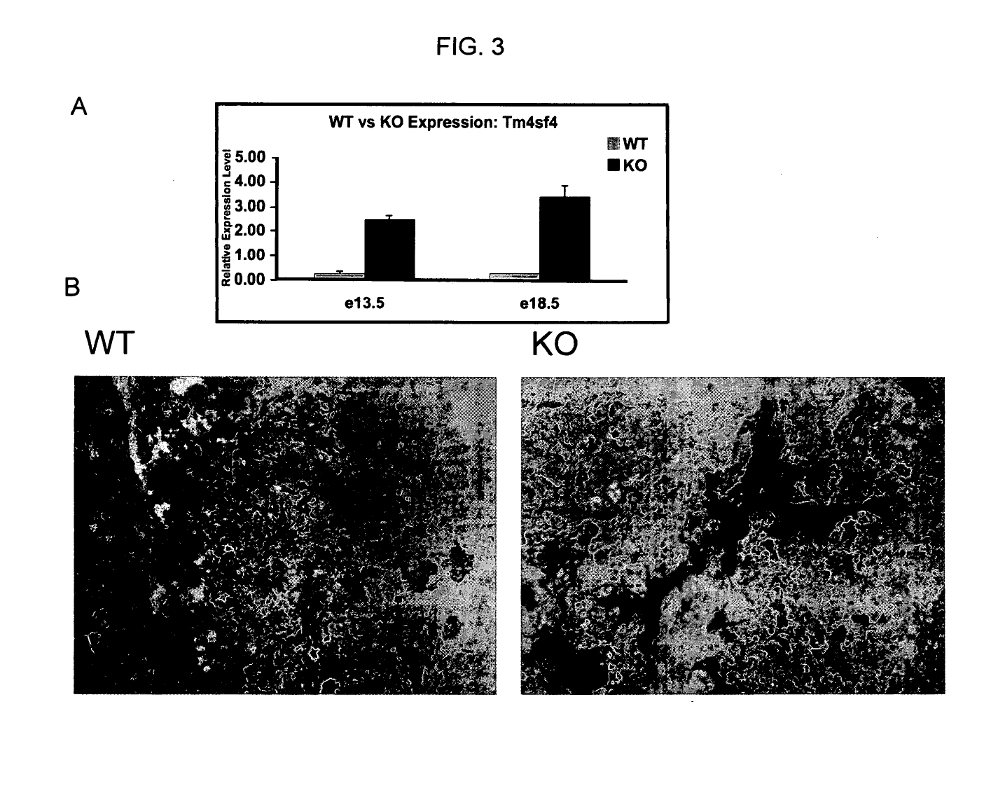

TM4SF4 was upregulated in Nkx2.2 mutant pancreas throughout gestation (FIG. 3A).

### Example 6

### RNA In Situ Analysis in Nkx2.2 Mutant Mice

Additionally, RNA in situ analysis of TM4SF4 expression in vivo was performed.

### Probe Synthesis

A full-length mouse cDNA clone was obtained from Open Biosystems (Huntsville, Ala., catalog #MMM1013-65619). The pCMV-Sport6:TM4SF4 plasmid was linearized with SalI, and T7 polymerase was used to create the antisense probe. The sense probe control was linearized with NotI, and Sp6 polymerase was used.

To make digoxigenin (DIG) or fluorescein labeled probes, DIG or fluorescein labeling mix (Roche, Nutley, N.J.) was mixed with the appropriate buffer (txn buffer, for example), linearized template DNA, SP6, T7 or T3 RNA polymerase (Roche, Nutley, N.J.), and incubated at 37° C. for 1 hour. RNA polymerase was then added, followed by additional incubation for 1 hour. After confirming that the probe has been added, the probe was precipitated with 1 μl glycogen (20 μg/ml, Roche, Nutley, N.J.), 7 μl 7.5M ammonium acetate, and 75 μl cold ethanol. This mixture was then incubated at −20° C. for 30 minutes. After collecting the pellet, a second precipitation was performed as above. The pellet was again collected and resuspended in sterile water.

### Pre-Hybridization of Sections

Tissue sections were fixed for 10 minutes in 4% PFA (paraformaldehyde, Sigma, St. Louis, Mo.)/PBS at room temperature, followed by three washes in PBS buffer for 3 minutes each. The sections were then digested in Proteinase K (1 μg/ml in 50 mM Tris pH 7.5, 5 mM EDTA). Incubation time varied according to the stage of the embryo: 2 minutes for e 10.5, 4 minutes for e 12.5, and 6 minutes for e 14.5.

Sections were then refixed for 5 minutes in 4% PFA/PBS at room temperature and followed again by three washes in PBS buffer for 3 minutes each. Then, the sections were acetylated for 10 minutes at room temperature in the appropriate buffer (to 300 ml water, add 4.08 ml triethanolamine, 0.534 ml HCL, 0.763 ml acetic anhydride). Then, three more washes in PBS buffer for 5 minutes each followed. Afterwards, approximately 500 μl hybridization buffer was added to each slide and incubated in a humidified chamber at 55° C. for 1-2 hours. The hybridization buffer consisted of 50% formamide, 5×SSC (pH 4.5, citric acid was used to adjust the pH), 50 μg/ml yeast tRNA (Sigma, St. Louis, Mo., catalog number R7876), 1% SDS, and 50 μg/ml heparin (Sigma, St. Louis, Mo., catalog number H3393). This hybridization buffer was removed just prior to the addition of probes.

### Hybridization:

Hybridization buffer containing probe at 1 ng/μl (1 μl of probe per 100 μl of hybridization buffer) was prepared. The probes were then heated at 80° C. for 5 minutes, followed by cooling at room temperature for 5 minutes. The probes were then added to sections and incubated overnight in an humidified chamber at 70° C.

### Washes and Antibody Addition

The slides were then submerged in prewarmed (70° C.) 5×SSC. Coverslips were removed and the slides were incubated on a rocker for 30 minutes at room temperature. The slides were then transferred to a prewarmed 0.2×SSC solution (pH 7) and incubated at 70° C. for 3 hours, followed by a 5 minute incubation at room temperature in 0.2×SSC. Then, the slides were transferred to MAB buffer (for 1 L of 5×MAB stock: maleic acid 58 g, NaCl 43.5 g, and pH adjusted to 7.5 with NaOH). Afterwards, the slides were transferred to blocking solution and incubated for at least 1 hour at room temperature. The blocking solution consisted of 2% blocking reagent (Roche, Nutley, N.J.), 10% heat inactivated sheep serum (Jackson Immunoresearch, West Grove, Pa., catalog number 013-000-121, inactivated at 56° C. for 30 minutes), 0.1% Tween-20 (Sigma, St. Louis, Mo., catalog number P1379) in 1×MAB buffer. Then, the blocking solution was replaced with fresh blocking solution containing anti-DIG-AP or anti-fluorescein antibody (Fab fragments; Roche, Nutley, N.J.) at a concentration of 1:5000. The slides were incubated overnight at 4° C.

### Washes and Staining

Slides were washed three times in 1×MAB with 0.1% Tween-20 for 15 minutes at room temperature, followed by washing in 0.1% Tween-20 for 20 minutes at room temperature. BM Purple (precipitating; Roche, Nutley, N.J.) with 0.1% Tween-20 were added to the slides, which were then wrapped in foil and incubated at room temperature until the desired signal was seen. The reaction was then stopped by transferring the slides to 1 mM EDTA in PBS. Afterwards, the slides were mounted with aqueous mounting media or counterstained and mounted with appropriate mounting media.

### Results

TM4SF4 was shown to be expressed in liver, intestine and pancreas in wild type mice. The TM4SF4 expression domain was shown to be expanded and the level of TM4SF4 expression was upregulated in the intestine and the pancreas of Nkx2.2 mutant mice.

### Example 7

### Cloning of Zebrafish homolog

The homolog of TM4SF4 cDNA was cloned from zebrafish (SEQ ID NO: 30).

The novel zebrafish coding sequence zgc:92479 on chromosome 22 with an accession number of BC078412 was PCR/TOPO cloned from a 48 hpf zebrafish cDNA library. The zTM4SF4 cDNA was then restriction digested from TOPO/pCRII with EcoRI and non-directionally cloned into pCS2 followed by restriction digestion and sequence verification for correct orientation. The following primers were used:

| | |
|---|---|
| Forward: | |
| 5′-ATCATGTGCTCTGGAAATTTCGCC | (SEQ ID No: 3) |
| Reverse: | |
| 5′-TTACTTTATTCCTTGCAGCAGCCG. | (SEQ ID No: 4) |

### Example 8

### Zebrafish In Situ Analysis

RNA in situ analysis was carried out to determine expression analysis of TM4SF4 in zebrafish. The protocol used was adapted from Thisse et al. (8).

### Preparation of Probe

A full-length probe was made after cloning the gene as described in Example 7.

DNA was prepared by linearizing pCS2-zTM4SF4 with BamHI, and the reaction was stopped using a mix of phenol/chloroform and then chloroform. DNA was then precipitated, washed with RNAse free 70% ethanol, and resupended in 10 mM Tris and 1 mM EDTA. Final concentration of the DNA was about 0.5 μg/μl.

The antisense RNA probe was prepared as follows. The DNA was incubated for 2 hours at 37° C. in transcription mix, which contained 1 μg linearized DNA, transcription buffer, NTP-DIG-RNA, 35 units of RNAse inhibitor, and 40 units of T7 RNA polymerase. The template DNA was then digested by adding 20 units of RNAse free DNAse for 15 minutes at 37° C. The reaction was stopped, and the RNA was precipitated for 30 minutes with the addition of 1 μl EDTA 0.5M pH 8, 2.5 μl LiCl 4M, 75 μl ethanol 100% at −70° C. The RNA was then centrifuged, washed with 70% ethanol, and resuspended in 20 μl sterile DEPC water.

### Fixation and Storage of Embryos

Zebrafish embryos were collected and treated as follows. The chorions were removed either by pronase treatment (for embryos older than 18 somites) or manually (for earlier stages). Then, the embryos were fixed in 4% paraformaldehyde (PFA) in PBS overnight at 4° C. Subsequently, the embryos were transferred into 100% methanol and stored at −20° C.

### Hybridization and Washing

The zebrafish embryos were rehydrated by successive incubations in 500 μL of 75% MeOH/25% PBS for 5 minutes, 50% MeOH/50% PBS for 5 minutes, and 25% MeOH and 75% PBS for 5 minutes. They were then washed in PBT (PBS/Tween20 1%) four times for 5 minutes each and digested with Proteinase K (Boehringer 1000144, stock: 10 mg/ml in H₂O) at the following conditions: (a) for 10 hpf (blastula and gastrula): 1 μl of PK stock in 5 ml of PBT, incubated for 30 seconds; (b) for 10-16 hpf (1-14 somites): 1 μl of PK stock in 5 ml of PBT, incubated for 1 minute; (c) 16-22 hpf (15-26 somites): 2 μl of PK stock in 2 ml of PBT, incubated for 2 minutes; and (d) 22-36 hpf: 2 μl of PK stock in 2 ml of PBT, incubated for 8 minutes. The embryos were then refixed in 4% PFA, 20 min at 4° C., followed by five washes in PBT for 5 minutes each.

The hybridization mix (50 ml) was prepared by mixing 25 ml of formamide 50-65% (depending on the stringency desired); 12.5 ml of 20×SSC, pH 7.5; 50 μl of Tween-20 0.1%; sufficient 1 M citric acid to adjust the pH to 6.0; heparin 50 μg/ml; tRNA 500 μg/ml; and water. The embryos were pre-hybridized in 800 μl of hybridization mix for 2 to 5 hours at 70° C. This prehybridization mix was then discarded, and replaced with 200 μl of pre-heated, 70° C. hybridization mix containing 100-200 ng of antisense RNA probe. This hybridization reaction was allowed to proceed overnight at 70° C.

The next day, the probes were removed. The embryos were rinsed with pre-warmed, 70° C. hybridization mix without the heparin and the tRNA, and washed three times with pre-warmed 70° C. hybridization mix (again without the heparin and the tRNA) for 20 minutes each. Then, the embryos were treated with 75% HM/25% 2×SSC at 70° C. for 15 min, 50% HM/50% 2×SSC at 70° C. for 15 minutes, 25% HM/75% 2×SSC at 70° C. for another 15 minutes, and finally 100% 2×SSC at 70° C. for 15 minutes. Depending on the stringency, the embryos were treated with 0.2×SSC (for normal stringency) or 0.05×SSC (for high stringency), twice for 30 minutes each at room temperature. Then, the embryos were washed in 75% 0.2× (or 0.05×) SSC/25% PBT at room temperature for 10 minutes, 50% 0.2× (or 0.05×) SSC/50% PBT at room temperature for 10 minutes, 25% 0.2× (or 0.05×) SSC/75% PBT at room temperature for 10 minutes, and finally PBT at room temperature for 10 minutes. The embryos were then incubated in a mixture of PBT, 2% sheep serum, 2 mg/ml BSA (Sigma, St. Louis, Mo., catalog number A-3294) at room temperature for at least 2 hours. This mixture was then incubated overnight with agitation at +4° C. in anti-DIG antibody solution, which contained pre-adsorbed anti-DIG (Boehringer 1 093 274, 1:1000 dilution (final concentration) in PBT), 2% sheep serum, and 2 mg/ml BSA.

The next day, the antiserum was removed, and the embryos were briefly rinsed once with PBT, and washed for six more times at room temperature for at least 1 hour each.

### Staining

Embryos in PBT were transferred to 12 or 24 well microtiter plates, washed three times for 10 minutes each with freshly made NTMT, which contained 100 mM of Tris, pH 9.5; 50 mM of MgCl₂; 100 mM of NaCl, 1% Tween 20. NTMT was then removed. The embryos were incubated with BM purple AP substrate (Roche, Nutley, N.J.) for 5 minutes on a rocker, then the mixture was placed on the bench and checked periodically for stain development. Once the desired stain was achieved, the reaction was stopped by washing twice for 5 minutes each in PBT. The embryos were then fixed for at least 2 hours at room temperature in 4% paraformaldehyde in PBS, or overnight at 4° C. The embryos were washed again in PBT twice for 5 minutes each. The embryos were then stored in PBT 4° C. until they were photographed or infiltrated and embedded.

### Embedding

The embryos were dehydrated through successive treatments of 50%, 70%, 90%, 95% ethanol, and then 100% ethanol twice for 5-10 minutes each. They were then treated with 50% JB4 catalyzed monomer A and 50% ethanol for several hours. Subsequently, the solution was replaced with 100% monomer A (infiltration solution) and the mixture was rocked at room temperature, followed by 2-3 washes for 30 minutes each until the embryos become transparent. The embryos were then placed in a mold, which contains an appropriate amount of embedding solution.

### Results

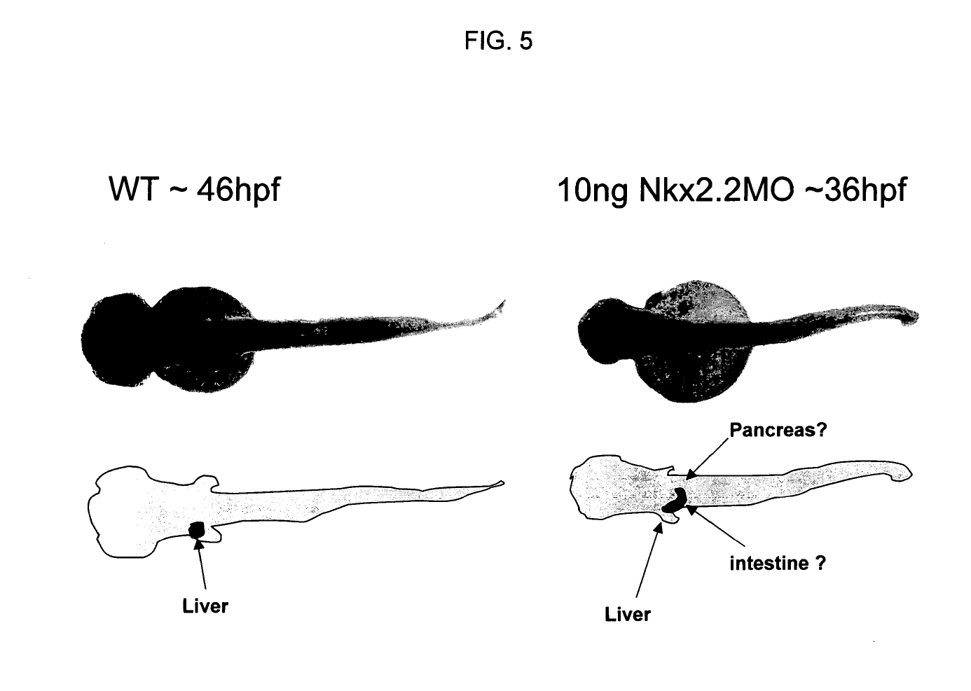

TM4SF4 was shown to be expressed in the livers of Zebrafish. TM4SF4 also appeared to be expressed in the pancreas and intestine (see FIG. 5).

### Example 9

### Zebrafish Knockdown Experiments

Knockdown of Nkx2.2 using morpholino technology (modified siRNAs) in zebrafish was performed.

For morpholino knockdown experiments, zebrafish matings were setup in the evening prior to day of injection. On the day of injection, embryos at 1 cell to 16 cell stages were collected from tanks and aligned in an agarose mold. A working stock solution of 2 ng/nl morpholino with rhodamine dextran (to visualize proper injection and amount injected) was loaded into pulled glass needles. Either 2.5 nl or 5 nl were injected, using a CO₂ pressure injector, into the yolks of the zebrafish embryos for 5 ng or 10 ng of morpholino, respectively. Embryos were incubated at 28.5° C. and staged appropriately. After 24 hours, embryos were raised in 1-phenyl-2-thiourea (PTU) in order to suppress pigmentation. Embryos were collected at the 48-hpf stage and de-chorionated prior to fixation. For TM4SF4, both a splice (exon2/intron2) and a translation (start site) blocking morpholino gave similar phenotypes with the splice blocker giving a stronger phenotype at lower doses (5 ng), which was used for all subsequent experiments. The translation blocking Nkx2.2 morpholino was directed against the 5′UTR and has previously been reported (S. Pauls et al. 2007).

The following morpholinos were used:

| | |
|---|---|
| MO Tm4sf4-atg: | |
| AATTTCCAGAGCACATGATTGAGTC | (SEQ ID NO: 5) |
| MO Tm4sf4-spl: | |
| GTTATTGTTTTTCTCACCGCAAATC | (SEQ ID NO: 6) |
| MOnk-5UTR: | |
| TGGAGCATTTGATGCAGTCAAGTTG | (SEQ ID NO: 7) |

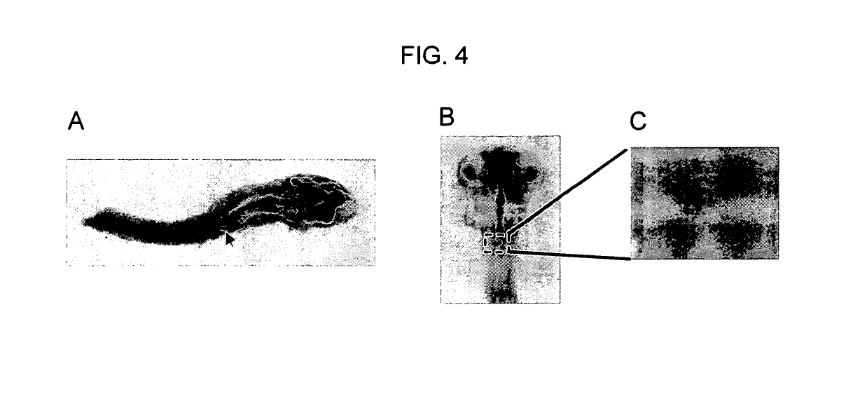

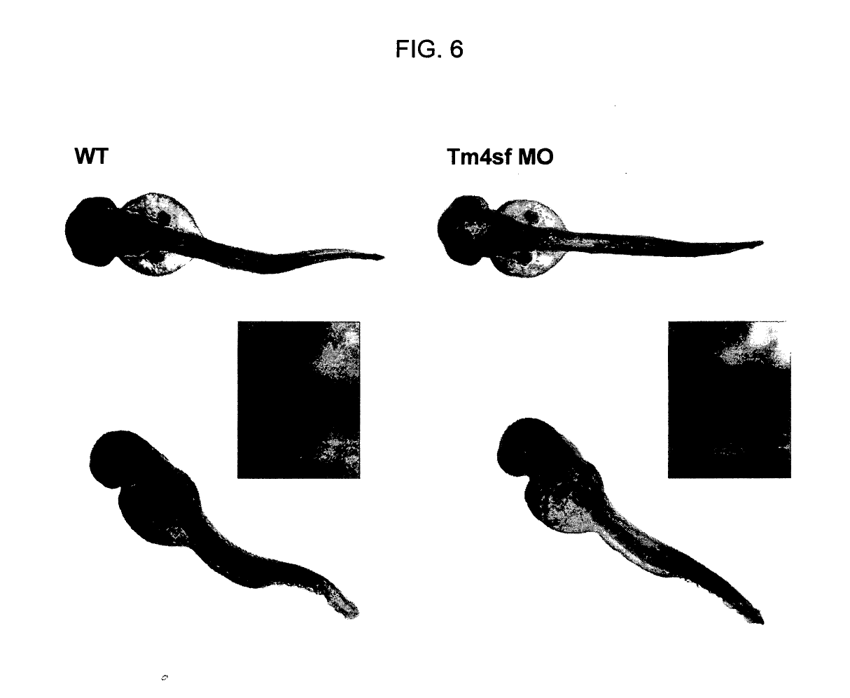

In this experiment, the TM4SF4 expression domain was expanded and the level of expression was upregulated. The affected tissues appeared to be pancreas and intestine. An expansion of the number of insulin producing cells (and perhaps insulin/cell) was observed. Glucagon producing cells might have also been expanded. (See, e.g., FIGS. 4-6.)

### Example 10

### Overexpression Analysis

Overexpression of TM4SF4 in immortalilzed alpha (glucagon) and beta (insulin) cell lines was performed. To overexpress mouse TM4SF4 in cell lines (bTC6, aTC1, mPAC L20, Panc1), full length TM4SF4 was cloned out of pCMV-Sport6 (BC010814, cat# mmm1013-65619, Open Biosystems) using XhoI and EcoRV restriction digests. The TM4SF4 insert was then ligated into the mammalian expression vector, pcDNA3. For lipid based transfections, 18 μg of pcDNA3-mTm4sf4, 65 μl FuGeneHD (3.5:1 ratio, Roche, Nutley, N.J. and Applied Sciences, Cederville, Ohio), and 500 μl serum-free media were incubated for 15 minutes to allow complex formation. Meanwhile, cells were trypsinized and seeded out at 60% confluency in 10 cm tissue culture treated plates. Immediately after seeding cells, DNA complexes were added dropwise to cells and allowed to incubate humidified at 37° C., 5% CO₂ for 72 hours prior to harvesting RNA for further analysis.

In this overexperssion system, little effect on hormone expression was seen.

### Example 11

### Generation of TM4SF4 Null Mice

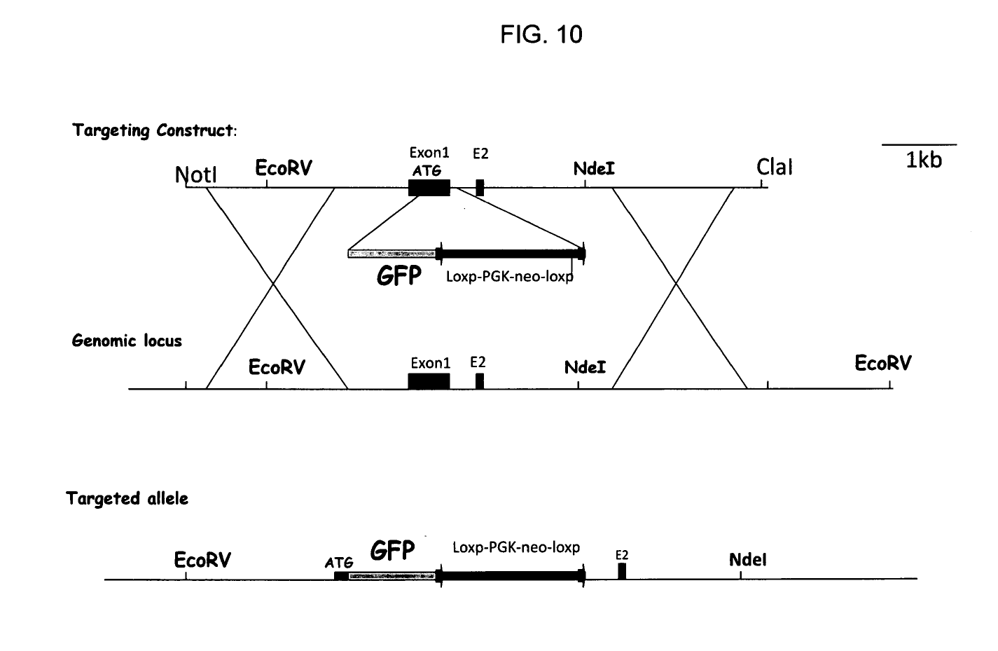

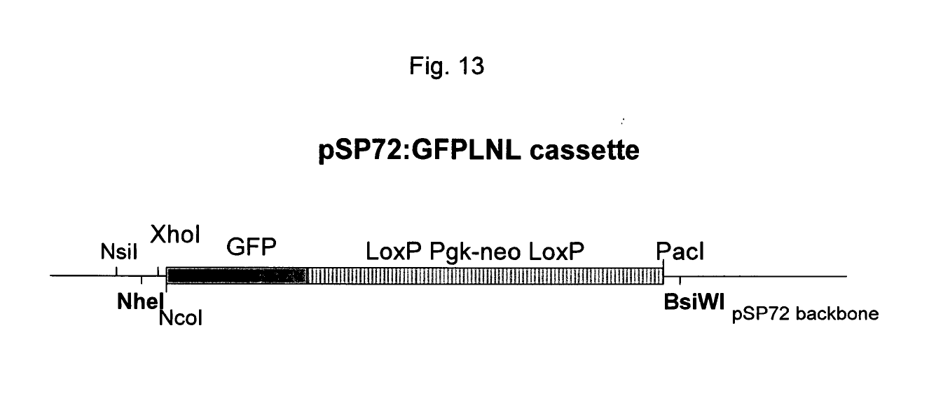

For the TM4SF4 knockout mouse, the GFP gene was introduced at the endogenous ATG start site of TM4SF4, effectively deleting the first exon of the Tm4SF4 gene. (See FIG. 10) The design goal was to delete a portion of exon1, but keep the endogenous ATG start site intact to drive GFP expression in place of Tm4sf4. A 129 background BAC clone, bMQ-165D4, containing the Tm4sf4 gene (Ensembl: ENSMUSG00000027801, MGI: 2385173) was purchased from Geneservice. A pair of oligos, (AGTCACTTTTTTTTCAAGAAATCTTTTATAAGAAT TAAACCCATGCTTAATATTAATAACTAGGCGGCCGCATTTAAATGGC (SEQ ID NO: 35) and CTACAGGCAGATCCAGAATATCTAGCTCTTTATCCCTTACAGGGGCAGCCAGA TTGAAGAACATCGATGATATCAGATCTGCC (SEQ ID NO: 36)), were designed to retrieve a 7249 base pair region of Tm4sf4 genomic DNA. These oligos have 65 base pairs homologous to the 5′ and 3′ end of the future Tm4sf4 knockout construct, respectively. These oligos also have a 21 base-pair homolog to pSP72 plasmid DNA (Promega, Madison, Wis.). A PCR reaction was performed using pSP72 as a template. After recombineering, colonies that were resistant to ampicillin were extracted. To initiate deletion/insertion, a pair of oligos flanking the region of deletion, (AAGCTGGTGGCGACGAGCCTTTGATCTCTGTGCTTTCCTGTGACCCCCCAGCATGTGT GCTCGAGACGTAGAAAGCCAGTCCGCAG (SEQ ID NO: 37) and CAAGACTCCACTTCCCAATATTCCTCCGAAGTACCAGACCTCATCCGAAAGGTGGCTTT TCGTACGCAAAATTCAGAAGAACTCGTCAAG (SEQ ID NO: 38)), were used to amplify a kanamycin resistant cassette. At this stage, the pSP72 Tm4sf4 plasmid should have a kanamycin cassette integrated into the Tm4sf4 first exon. The final Tm4sf4GFPLNL construct was generated by replacing the kanamycin cassette (cut with restriction enzymes Nhe and BsiWl) with a GFPLNL cassette (similarly cut with restriction enzymes Nhe and BsiWl). A map of the final construct containing GFP-LoxP Pgk-neo LoxP (SEQ ID NO:63) is shown in FIG. 13. The construct DNA was linearized by ClaI digestion, and electroporated into 129 ES cells. Colonies were selected for Neomycin resistance. After southern screening, 3 out of 296 ES cell clones were identified. One pure clone was used for injecting into C57BL/6 blastocysts. Four male chimeras with greater than 95% agouti were mated with Black Swiss females, 3 of the males gave germline transmission.

### Example 12

### siRNA and Antibodies Against TM4SF4

Eight siRNAs against TM4SF4 (4 against human and 4 against mice) were generated. The sequences of these siRNAs are listed in Table 3 below.

TABLE 3
Human siRNAs
siRNA hTm4 idt4

| | |
|---|---|
| Sense | GATCCCCggctgtcatcatggctagggttTTCAAGAGA |
| | aaccctagccatgatgacagccTTTTTGGAAA |
| | (SEQ ID NO: 10) |
| Antisense | AGCTTTTCCAAAAAggctgtcatcatggctagggttTC |
| | TCTTGAAaaccctagccatgatgacagccGGG |
| | (SEQ ID NO: 11) |

siRNA hTm4 idt5

| | |
|---|---|
| Sense | GATCCCCgagaaactaagaccaatttctgtTTCAAGAG |
| | AacagaaattggtcttagtttctcTTTTTGGAAA |
| | (SEQ ID NO: 12) |
| Antisense | AGCTTTTCCAAAAAgagaaactaagaccaatttctgtT |
| | CTCTTGAAacagaaattggtcttagtttctcGGG |
| | (SEQ ID NO: 13) |

siRNA hTm4 dh6

| | |
|---|---|
| Sense | GATCCCCGAAAAGTGATAGATGACAATTCAAGAGATTG |
| | TCATCTATCACTTTTCTTTTTGGAAA |
| | (SEQ ID NO: 14) |
| Antisense | AGCTTTTCCAAAAAGAAAAGTGATAGATGACAATCTCT |
| | TGAATTGTCATCTATCACTTTTCGGG |
| | (SEQ ID NO: 15) |

siRNA hTm4 ob899

| | |
|---|---|
| Sense | GATCCCCAGCTGGATACTCGTTTATCATTTCAAGAGAA |
| | TGATAAACGAGTATCCAGCTTTTTTGGAAA |
| | (SEQ ID NO: 16) |
| Antisense | AGCTTTTCCAAAAAAGCTGGATACTCGTTTATCATTCT |
| | CTTGAAATGATAAACGAGTATCCAGCTGGG |
| | (SEQ ID NO: 17) |

Mouse siRNAs
siRNA mTm4 ob184

| | |
|---|---|
| Sense | GATCCCCgccatattgtctctttgtataTTCAAGAGAt |
| | atacaaagagacaatatggcTTTTTGGAAA |
| | (SEQ ID NO: 20) |
| Antisense | AGCTTTTCCAAAAAgccatattgtctctttgtataTCT |
| | CTTGAAtatacaaagagacaatatggcGGG |
| | (SEQ ID NO: 21) |

siRNA mTm4 idt7

| | |
|---|---|
| Sense | GATCCCCgcctgtttgactaatgtgtctggTTCAAGAG |
| | AccagacacattagtcaaacaggcTTTTTGGAAA |
| | (SEQ ID NO: 22) |
| Antisense | AGCTTTTCCAAAAAgcctgtttgactaatgtgtctggT |
| | CTCTTGAAccagacacattagtcaaacaggcGGG |
| | (SEQ ID NO: 23) |

siRNA mTm4 ob188

| | |
|---|---|
| Sense | GATCCCCccacgatggtgattatctaaaTTCAAGAGAg |
| | cctgtttgactaatgtgtctggTTTTGGGAAA |
| | (SEQ ID NO: 24) |
| Antisense | AGCTTTTCCAAAAAccacgatggtgattatctaaaTCT |
| | CTTGAAgcctgtttgactaatgtgtctggGGG |
| | (SEQ ID NO: 25) |

siRNA mTm4 dh1

| | |
|---|---|
| Sense | GATCCCCgcataactcaagtggataaTTCAAGAGAtta |
| | tccacttgagttatgcTTTTTGGAAA |
| | (SEQ ID NO: 26) |
| Antisense | AGCTTTTCCAAAAAgcataactcaagtggataaTCTCT |
| | TGAAttatccacttgagttatgcGGG |
| | (SEQ ID NO: 27) |

A peptide from the extracellular domain of Tm4sf4 was used to generate polyclonal antibodies against Tm4sf4. The peptide (CGTWGYPFHDGDYLKD, SEQ ID NO: 34) was synthesized and activated with Keyhole limpet hemocyanin (KLH). The modified peptide was then sent to Covance (Princeton, N.J.) for the generation of antibodies. Two rabbits were immunized, boosted and bled to generate antiserum.

More GST fusion constructs containing extracellular domains of TM4SF4 may be created, and these constructs will be used to generate antibodies according to the method disclosed above or other methods known in the art.

These siRNAs and antibodies will be used to block the activity of the TM4SF4 protein. siRNA blocks the production of the protein, and antibodies block the function of the protein. It is expected that such siRNAs and antibodies will result in an increase in the number and/or function of β-cells and/or an increase in insulin levels.

Anti-TM4SF4 antibodies and/or siRNAs will be used in primary mouse embryonic pancreas cultures to determine whether insulin and beta cell production may be enhanced ex vivo. Additionally, anti-TM4SF4 antibodies and/or siRNAs will be used in mouse and human ES cells for pancreas differentiation cultures to assess whether loss of TM4SF4 can cause more efficient production of insulin-producing β-cells. Furthermore, such constructs will be used in postnatal primary exocrine, islet and/or ductal cultures (mouse and human) to see whether the differentiation of insulin producing cells may be promoted from a rare progenitor population.

If any of these ex vivo or cell line approaches are successful, attempts will be made to deliver the siRNAs to the pancreas in vivo (mouse) to determine the ability to promote insulin-cell production at several stages of development and postnatally.

### Example 13

### Small Molecule Modulators

A screen for small molecule modulators will depend on the success of TM4SF4 to promote beta cell production in the mouse and/or human ES cell differentiation system. For the purposes of the screen, mouse and human ES cells that carry an Insulin:GFP transgene will be used to perform a high throughput screen. The screen is based on Dr. Doug Melton's (Harvard) high throughput screen in an ES cell as well as a definitive endoderm differentiation protocol to identify small molecules that promote the formation of endoderm, demonstrating the feasibility of this approach.

### Example 14

### Quantitative Real Time PCR Analysis in Zebrafish Treated with Tm4sf4 Morpholinos

Fourteen wild-type and Tm4sf4 morphants were collected and stored in RNAlater (Ambion) at 4° C. Prior to harvesting RNA, heads and tails below the yolk sac were removed in order to concentrate pancreas-expressing RNA. All 14 embryos from each condition were pooled into one sample. Samples were then homogenized using a Dounce homogenizer, and RNA was collected using an RNeasy Micro Kit (Qiagen). cDNA was made from 1.25 μg of RNA for each sample using Superscript III Kit (Invitrogen Life Technologies) according to the manufacturer's instructions. Each quantitative PCR reaction contained 1 μg of cDNA and genes of interest were normalized to β-actin expression. The primers used for quantitative PCR reaction are set forth in Table 4 below.

TABLE 4

| | | SEQ ID |
|---|---|---|
| Name | sequence | NO |
| β-actin | cat cag ggt gtc atg gtt ggt | 39 |
| forward | | |
| β-actin | tct ctt gct ctg agc ctc atc a | 40 |
| reverse | | |
| β-actin probe | tgg gac aga aag aca gct a | 41 |
| Ghrelin | tcc tca gtc cga ctc aga aac c | 42 |
| forward | | |
| Ghrelin | gct tct ctt ctg ccc act ctt g | 43 |
| reverse | | |
| Ghrelin probe | agg gtc gaa ggc ca | 44 |
| Glucagon | aag cga gga gac gat cca aa | 45 |
| forward | | |
| Glucagon | tcc aac aca cac cag caa atg | 46 |
| reverse | | |
| Glucagon probe | aca ttt cat atc atc tca tcc | 47 |
| Insulin | gag ccc ctt ctg ggt ttc c | 48 |
| forward | | |
| Insulin | aag tca gcc acc tca gtt tcc t | 49 |
| reverse | | |
| Insulin probe | tcc tcc taa atc tgc c | 50 |
| Nkx2.2a | aac cac gga cag cat cca at | 51 |
| forward | | |
| Nkx2.2a | ttt gcg gac gtg tct tga ga | 52 |
| reverse | | |
| Nkx2.2a probe | tca tta cac ggc ctg tcc gcg aa | 53 |
| Pdx1 forward | cac acg cac gca tgg aaa | 54 |
| Pdx1 reverse | tcc tcg gcc tcg acc ata t | 55 |
| Pdx1 probe | cag tgg aca ggc cct | 56 |
| Somatostatin | gcc aaa ctc cgc caa ctt c | 57 |
| forward | | |
| Somatostatin | ctg gcg agt tcc tgt ttt cc | 58 |
| reverse | | |
| Somatostatin | atc tct cct cag ccc tg | 59 |
| probe | | |

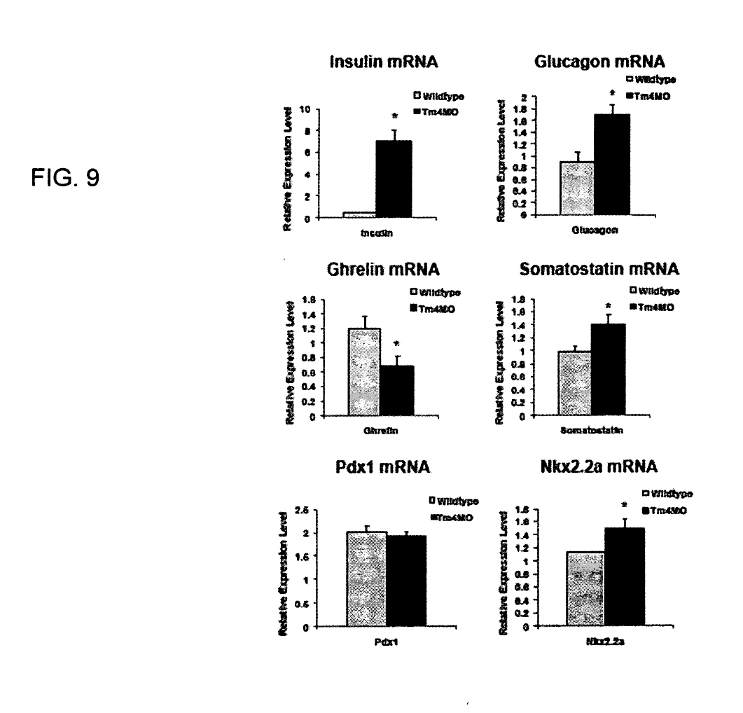

The results are shown in FIG. 9. The expression of insulin, glucagon, somatostatin, and Nkx2.2 was significantly higher in Tm4sf4 morphants. Ghrelin expression was significantly lower in Tm4sf4 morphants.

### Example 15

### Quantitative Real Time PCR Analysis and Immunostaining in Tm4sf^{-/-} Mice

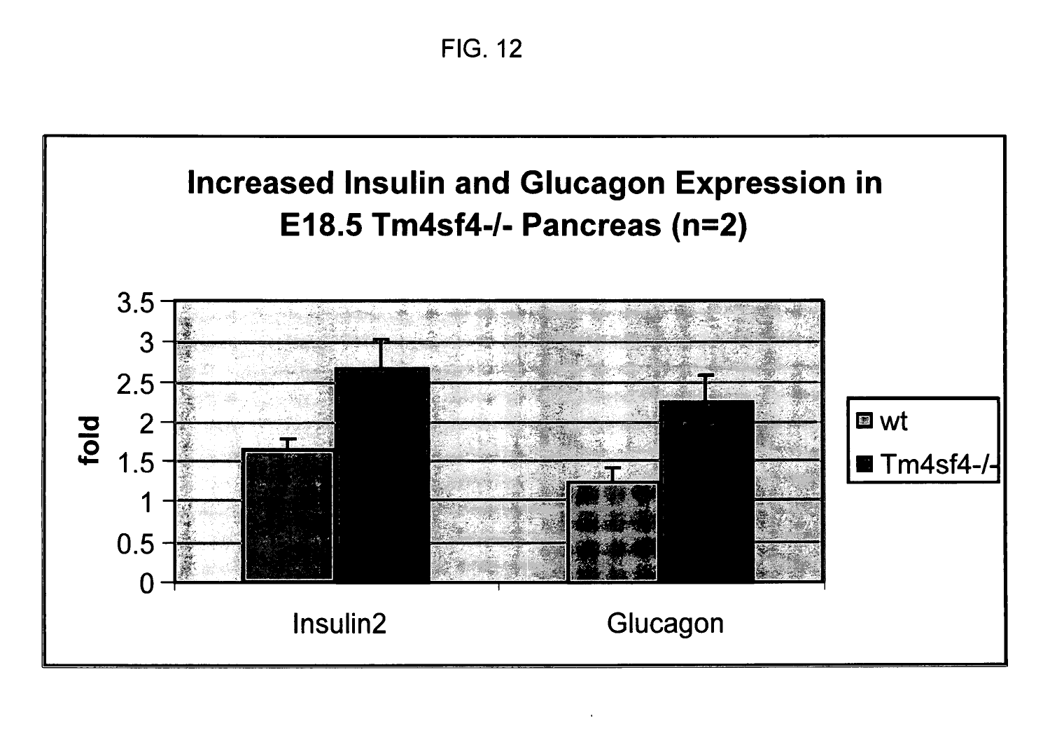

Total RNA was harvested from e 18.5 whole mouse pancreata using the RNeasy Micro Kit (Qiagen). Two Tm4sf4 mutant and two wild-type littermates were used for this experiment. For each sample, 0.5 μg to 1 μg mRNA was converted to cDNA using the Superscript III Kit (Invitrogen Life Technologies) Q-RTPCR was performed using custom and pre-designed Taqman primer/probes (Applied Biosystems) and all probes were fluorescently labeled at the 5′-end with 6-carboxyfluorescein (6FAM) and with a minor groove binder (MGB) non-fluorescent quencher at the 3′-end. Insulin and glucagon expression levels were assed using Taqman Gene Expression Assay (Applied Biosystems, Foster City, Calif.; #Mm00801712_m1 for insulin, and #Mm00731595_gH for glucagon). mRNA expression for each sample was normalized to expression of the ubiquitous metabolic control gene, cyclophilin B. The forward primer used for assessing cyclophilin B expression level was GCAAAGTTCTAGAGGGCATGGA (SEQ ID NO: 60); the reverse primer used was CCCGGCTGTCTGTCTTGGT (SEQ ID NO: 61); and the probe used was TGGTACGGAAGGTGGAG (SEQ ID NO: 62). The results are show in FIG. 12. Both insulin mRNA and glucagon mRNA expression levels were increased in Tm4sf4^{-/-} mice.

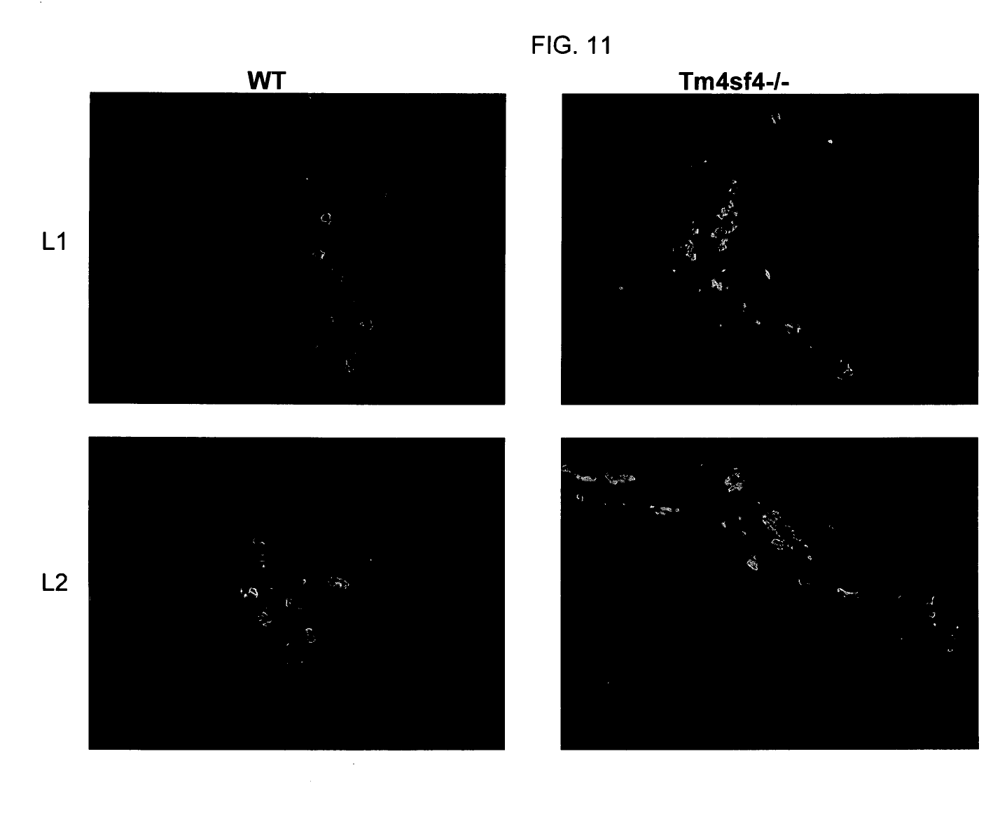

Immunofluorescence was performed on e15.5 whole embryo frozen 8 μm sections that were fixed for 3 hours with 4% paraformaldehyde. Antibodies used consisted of guinea pig anti-insulin (1:1000, Dako Denmark A/S) and mouse anti-glucagon (1:1000, Sigma). Secondary antibodies (Jackson ImmunoResearch Laboratories, Inc., West Grove, Pa.) were used against individual species, all raised in donkey, labeled with either Cy2 or Cy5, and used at 1:300. DAPI (Invitrogen Life Technologies) was used at 1:1000 and incubated for 30 minutes. Confocal images were taken on a Zeiss META LSM 510. The results are shown in FIG. 11. These experiments showed that insulin and glucagon expression levels are increased in Tm4sf4^{-/-} mice.

### CITED DOCUMENTS

All documents cited in this application are hereby incorporated by reference as if recited in full herein.
1. L. Sussel et al., Development 125: 2213-2221 (1998).
2. Tusher, VG, Tibshirani, R & Chu, G Significance analysis of microarrays applied to the ionizing radiation response. PNAS 98:5116-21 (2001).
3. Storey, J “A direct approach to false discovery rates”. J. Roy. Stat. Soc. Ser. B, 64:479-498 (2002).
4. Lonnstedt, I. & Speed, T. Replicated Microarray Data. Statistica Sinica 12, 31-46 (2002).
5. Smyth, G. K. Linear models and empirical bayes methods for assessing differential expression in microarray experiments. Statistical Applications in Genetics and Molecular Biology, 3:Article 3 (2004).
6. Bolstad, B. M., Irizarry R. A., Astrand, M, and Speed, T. P. A Comparison of Normalization Methods for High Density Oligonucleotide Array Data Based on Bias and Variance. Bioinformatics 19(2): 185-193 (2003).
7. Suzuki R, Shimodaira H. Pvclust: an R package for assessing the uncertainty in hierarchical clustering. Bioinformatics. 22(12):1540-2 (2006).
8. Thisse, C., Thisse, B., Schilling, T. F., and Postlethwait, J. H. Structure of the zebrafish snaill gene and its expression in wild-type, spadetail and no tail mutant embryos. Development 119, 1203-1215 (1993).
9. Chao et al., Genetic identification of a novel NeuroD1 function in the early differentiation of islet alpha, PP and epsilon cells. Dev Biol. 312(2):523-32 (2007).

Although illustrative embodiments of the present invention have been described herein, it should be understood that the invention is not limited to those described, and that various other changes or modifications may be made by one skilled in the art without departing from the scope or spirit of the invention.

What is claimed is:

1. A method for modulating a β-cell population comprising contacting an islet progenitor cell population with an amount of a transmembrane 4 superfamily member 4 (TM4SF4) modulator or a modulator of a TM4SF4 homolog, which is sufficient to modulate the production of β-cell population.

2. The method according to claim 1, wherein the TM4SF4 homolog is a human intestinal and liver tetraspan membrane protein (il-TMP).

3. The method according to claim 1, wherein the TM4SF4 modulator inhibits TM4SF4 transcription, translation, or function.

4. The method according to claim 1, wherein the TM4SF4 modulator is selected from the group consisting of nucleic acids, polypeptides, polysaccharides, small molecules, and combinations thereof.

5. The method according to claim 4, wherein the modulator is selected from the group consisting of a fusion protein, an antibody, an antibody mimetic, a domain antibody, a targeted aptamer, an RNAi, an siRNA, an shRNA, an antisense sequence, and combinations thereof.

6. The method according to claim 1, wherein the modulator acts upstream of TM4SF4.

7. The method according to claim 1, wherein the modulator acts downstream of TM4SF4.

8. A pharmaceutical composition comprising a pharmaceutically acceptable carrier and a TM4SF4 modulator or a modulator of a TM4SF4 homolog, which modulator is present in the composition in an amount sufficient to modulate production of a β-cell population when administered to a patient in need thereof.

9. The pharmaceutical composition according to claim 8, wherein the TM4SF4 homolog is a human il-TMP.

10. The pharmaceutical composition according to claim 8, wherein the TM4SF4 modulator inhibits TM4SF4 transcription, translation, or function.

11. The pharmaceutical composition according to claim 8, wherein the TM4SF4 modulator is selected from the group consisting of nucleic acids, polypeptides, polysaccharides, small molecules, and combinations thereof.

12. The pharmaceutical composition according to claim 10, wherein the modulator is selected from the group consisting of a fusion protein, an antibody, an antibody mimetic, a domain antibody, a targeted aptamer, an RNAi, an siRNA, an shRNA, an antisense sequence, and combinations thereof.

13. The pharmaceutical composition according to claim 8, wherein the modulator acts upstream of TM4SF4.

14. The pharmaceutical composition according to claim 8, wherein the modulator acts downstream of TM4SF4.

15. The pharmaceutical composition according to claim 8, wherein the TM4SF4 modulator or the modulator of the TM4SF4 homolog increases production of the 13-cell population.

16. A method for expanding a β-cell population comprising contacting an islet progenitor cell population comprising a β-cell with a TM4SF4 modulator or a modulator of a TM4SF4 homolog for a period of time sufficient to expand the number of β-cells in the population.

17. The method according to claim 16, wherein the TM4SF4 homolog is a human il-TMP.

18. The method according to claim 16, wherein the contacting step comprises administering the TM4SF4 modulator to a patient.

19. The method according to claim 16, wherein the contacting step comprises ex vivo administration of the TM4SF4 to the cell population.

20. The method according to claim 19, wherein the population of islet progenitor cell population is obtained from a pancreas.

21. The method according to claim 16, wherein the TM4SF4 modulator inhibits TM4SF4 transcription, translation, or function.

22. The method according to claim 16, wherein the TM4SF4 modulator is selected from the group consisting of nucleic acids, polypeptides, polysaccharides, small molecules, and combinations thereof.

23. The method according to claim 22, wherein the modulator is selected from the group consisting of a fusion protein, an antibody, an antibody mimetic, a domain antibody, a targeted aptamer, an RNAi, an siRNA, an shRNA, an antisense sequence, and combinations thereof.

24. The method according to claim 16, wherein the modulator acts upstream of TM4SF4.

25. The method according to claim 16, wherein the modulator acts downstream of TM4SF4.

26. A method for treating a disease associated with a loss of β-cells comprising administering to a patient in need thereof an expanded β-cell population obtained using the method of claim 19.

27. The method according to claim 26, wherein the expanded β-cell population is from an autologous source.

28. The method according to claim 26, wherein the expanded β-cell population is from an heterologous source.

29. A method for treating a disease associated with a loss of β-cells comprising administering to a patient in need thereof a pharmaceutical composition according to claim 8.

30. The method according to claim 29, wherein the disease is diabetes.

31. The method according to claim 29, wherein the disease is type I diabetes.

32. A method of increasing the production of β-cells from a cell population comprising contacting the cell population with a TM4SF4 inhibitor or an inhibitor of a TM4SF4 homolog.

33. The method according to claim 32, wherein the TM4SF4 homolog is a human il-TMP.

34. The method according to claim 32, wherein the cell population is selected from embryonic stem cells or adult progenitor cells.

35. The method according to claim 32, wherein the cell population comprises islet progenitor cells.


SEQUENCE LISTING (66 sequences)

SEQ ID NO: 1 - protein, 202 aa, Homo sapiens
MCTGGCARCLGGTLIPLAFFGFLANILLFFPGGKVIDDNDHLSQEIWFFGGILGSGVLMIFPALVFLGLKNNDCCGCCGNEGCGKRFAMFTSTIFAVVGFLGAGYSFIISAISINKGPKCLMANSTWGYPFHDGDYLNDEALWNKCREPLNVVPWNLTLFSILLVVGGIQMVLCAIQVVNGLLGTLCGDCQCCGCCGGDGPV

SEQ ID NO: 2 - DNA, 1428 bp, Homo sapiens
cttcaggtcagggagaatgtataaatgtccattgccatcgaggttctgctatttttgagaagctgaagcaactccaaggacacagttcacagaaatttggttctcagccccaaaatactgattgaattggagacaattacaaggactctctggccaaaaacccttgaagaggccccgtgaaggaggcagtgaggagcttttgattgctgacctgtgtcgtaccaccccagaatgtgcactgggggctgtgccagatgcctgggggggaccctcattccccttgctttttttggcttcctggctaacatcctgttattttttcctggaggaaaagtgatagatgacaacgaccacctttcccaagagatctggtttttcggaggaatattaggaagcggtgtcttgatgatcttccctgcgctggtgttcttgggcctgaagaacaatgactgctgtgggtgctgcggcaacgagggctgtgggaagcgatttgcgatgttcacctccacgatatttgctgtggttggattcttgggagctggatactcgtttatcatctcagccatttcaatcaacaagggtcctaaatgcctcatggccaatagtacatggggctaccccttccacgacggggattatctcaatgatgaggccttatggaacaagtgccgagagcctctcaatgtggttccctggaatctgaccctcttctccatcctgctggtcgtaggaggaatccagatggttctctgcgccatccaggtggtcaatggcctcctggggaccctctgtggggactgccagtgttgtggctgctgtgggggagatggacccgtttaaacctccgagatgagctgctcagactctacagcatgacgactacaatttcttttcataaaacttcttctcttcttggaattattaattcctatctgcttcctagctgataaagcttagaaaaggcagttattccttctttccaaccagctttgctcgagttagaattttgttattttcaaataaaaaatagtttggccacttaacaaatttgatttataaatctttcaaattagttcctttttagaatttaccaacaggttcaaagcatacttttcatgatttttttattacaaatgtaaaatgtataaagtcacatgtactgccatactacttctttgtatataaagatgtttatatctttggaagttttacataaatcaaaggaagaaagcacatttaaaatgagaaactaagaccaatttctgtttttaagaggaaaaagaatgattgatgtatcctaagtattgttatttgttgtctttttttgctgccttgcttgagttgcttgtgactgatcttttgaggctgtcatcatggctagggttcttttatgtatgttaaattaaaacctgaattcagaggtaacgt

SEQ ID NO: 3 - DNA, 24 bp, Artificial
  Synthetic Construct; cloning primer_forward
atcatgtgctctggaaatttcgcc

SEQ ID NO: 4 - DNA, 24 bp, Artificial
  Synthetic Construct; cloning primer_reverse
ttactttattccttgcagcagccg

SEQ ID NO: 5 - DNA, 25 bp, Artificial
  Synthetic construct; MO Tm4sf4-atg
aatttccagagcacatgattgagtc

SEQ ID NO: 6 - DNA, 25 bp, Artificial
  Synthetic Construct; MO Tm4sf4-spl
gttattgtttttctcaccgcaaatc

SEQ ID NO: 7 - DNA, 25 bp, Artificial
  Synthetic Construct; MOnk-5UTR
tggagcatttgatgcagtcaagttg

SEQ ID NO: 8 - DNA, 1576 bp, Danio rerio
gtgcactccttactttcatttggagatatttcattaactgtgcggtgtctcaacgcaagctgcaccacttaccctcttccgtcagccgccagaggattgtcaaactggtgcagcaacttgactgcatcaaatgctccagaacatgtcgttgaccaacacaaagacgggcttttcagtaaaggacattttggacctccctgatactaacgatgaagaaggatctatcaccggcaccgaggaagatacggagggttctgaggcgactaaaacgcctggagtgttagtgcaaagtcctttagagaatgttcaaaacctgcctttaaagaatccattttatgataatagcgacaatccttatactagatggctcgcaaccacggacagcatccaatactcattacacggcctgtccgcgaactctcaagacacgtccgcaaaatctccggaaccttcagctgatgaatcgccggacaatgacaaggaaacttcaagcaacggcagcgactctggtaagaagcgcaaaaggagggtgctcttttccaaggcacaaacttacgaactcgagcgccggtttaggcaacagagatatctgtcagccccggagagggagcatctcgccagtttgattcgcctgactccaacccaagtgaaaatctggttccagaaccaccgatataaaatgaagagagcccgggcggagaaaggtatggaagtgacccatctcccttctcctaggcgggtggccgtgcctgtgttagtcagagacggcaaaccttgccatacgctaaaagctcaggacttggcagctacttttcaggctggaattccgttctctgcgtatagcgcccagtctctccagcacatgcaatataacgcgcattacagcgccgccactacaccacagttcccaacagcacatcacttggtgcaaacgcaacagtggacttggtgaacaaatactgacttgcaatgcagggttgcattttaaagaccgtggtagggagctttccaaacaaaaatacaagactttttatttttgcagcttgaccctttctcaccaatttattttggggattgacgtgtgcgccatgtttacatgttttcgttaagaaaaccgggtcaagcctctccccaaatttaagaatgttaaagctcttgtgaaattttgtaaagtatgtttgtgtgttgtagaaatgtttttcttttgtccttcgtgttatacgcacgtgcaaaaccactttatgattatagaaaattttatttctttcttttacaatggaccgaatatatttatggccatgaataaacactggcaatccttagcttatttcattggtttgttttatatgtaaatattttgtgataactgtttgcaatattatgtttgaccgtccagtaagctatatcctaaatatatgccatatttagggaagatggaacatcattgtcgacaatatcccttctgttaatttcagcaggtaacaacgcgcgctggcgagggaagtctacagttctttttattaaaacttttaataaaattatgtgtttcagaaaaaaaaaaaaaaaaaaaaaaaaa

SEQ ID NO: 9 - protein, 269 aa, Danio rerio
MSLTNTKTGFSVKDILDLPDTNDEEGSITGTEEDTEGSEATKTPGVLVQSPLENVQNLPLKNPFYDNSDNPYTRWLATTDSIQYSLHGLSANSQDTSAKSPEPSADESPDNDKETSSNGSDSGKKRKRRVLFSKAQTYELERRFRQQRYLSAPEREHLASLIRLTPTQVKIWFQNHRYKMKRARAEKGMEVTHLPSPRRVAVPVLVRDGKPCHTLKAQDLAATFQAGIPFSAYSAQSLQHMQYNAHYSAATTPQFPTAHHLVQTQQWTW

SEQ ID NO: 10 - DNA, 70 bp, Artificial
  Synthetic construct; siRNA hTm4 idt4, sense
gatccccggctgtcatcatggctagggttttcaagagaaaccctagccatgatgacagcctttttggaaa

SEQ ID NO: 11 - DNA, 70 bp, Artificial
  Synthetic Construct; siRNA hTm4 idt4, antisense
agcttttccaaaaaggctgtcatcatggctagggtttctcttgaaaaccctagccatgatgacagccggg

SEQ ID NO: 12 - DNA, 72 bp, Artificial
  Synthetic Construct; siRNA hTm4 idt5_sense
gatccccgagaaactaagaccaatttctgtttcaagagaacagaaattggtcttagtttctctttttggaaa

SEQ ID NO: 13 - DNA, 72 bp, Artificial
  Synthetic Construct; siRNA hTm4 idt5_antisense
agcttttccaaaaagagaaactaagaccaatttctgttctcttgaaacagaaattggtcttagtttctcggg

SEQ ID NO: 14 - DNA, 64 bp, Artificial
  Synthetic Construct; siRNA hTm4 dh6_sense
gatccccgaaaagtgatagatgacaattcaagagattgtcatctatcacttttctttttggaaa

SEQ ID NO: 15 - DNA, 64 bp, Artificial
  Synthetic Construct; siRNA hTm4 dh6_antisense
agcttttccaaaaagaaaagtgatagatgacaatctcttgaattgtcatctatcacttttcggg

SEQ ID NO: 16 - DNA, 68 bp, Artificial
  Synthetic Construct; siRNA hTm4 ob899_sense
gatccccagctggatactcgtttatcatttcaagagaatgataaacgagtatccagcttttttggaaa

SEQ ID NO: 17 - DNA, 68 bp, Artificial
  Synthetic Construct; siRNA hTm4 ob899_antisense
agcttttccaaaaaagctggatactcgtttatcattctcttgaaatgataaacgagtatccagctggg

SEQ ID NO: 18 - DNA, 2026 bp, Mus musculus
  misc_feature (2019)..(2019): n is a, c, g, or t
cggagccggagctgacggcaccttggcacctctcctggagttacaaactgaggccgcgcggcgctgggcgcgcaggccgccagtcacagcctacatttctgcgtgctttccgagaagagagaggcaccgggtgggctttatttttttttcccctttcccttttccccccacagtgtcctctcattttaaataataattatcccaataattaaaacccatcccccatccctcccccccattcctttccttaaacccccctcccccgcccgctggggctggggagagccacgaattgaccaagtgaggctacaactttgtggcataaattgcggggtccggaaccatgtcgctgaccaacacaaagacggggttttcagtcaaggacatcttggaccttccggacaccaacgatgaagacggctcggtggccgaagggccagaggaggagagcgaagggccggagcccgccaagagggccgggccgctggggcagggcgccctggacgctgtgcagagcctgccccttaagagccctttctacgacagcagcgacaacccctacactcgctggctggccagcaccgagggcctccaatactccctgcacgggctggcggccagcgctcccccccaagactcgagctccaagtccccagagccctcggctgacgagtcaccggacaatgacaaggagacccagggcggcgggggggacgcaggcaagaagcggaagcgccgagtgctcttctccaaagcgcagacctacgagctggagcggcgcttccggcagcagcggtacctgtcggcgcccgagcgcgagcacctggccagcctcatccgtctcacgccgacacaggtcaagatctggttccagaaccatcgctacaagatgaaacgtgcccgggcggagaaaggtatggaggtgacgcctctgccctcgccgcgccgtgtggcagtgccggtcttggtcagggacggcaaaccgtgccacgcgctcaaagcccaggacctggcagccgccaccttccaggcaggcatccccttttccgcctacagcgcgcagtcgctgcagcacatgcagtacaacgcccagtacagctcggccagcaccccccagtacccgacagcacaccccctggtccaggcccagcagtggacttggtgagcgccgcccctccaagactggaggccttaggccctggccccaccccagcggcgccggtggcgaggaggactcgatccttaccacggttgttattattattattataattattattaaagagtcgagtctgactttcgactcagttgaggaggcacagggaggttgcctgtgcctccccgagtggcagattccattgactctgccccatcgctctcccctttgaacttttggagaggatagaactctaagccgtgtttacagaatgttgtgcagctttgcttctttgcctctctctccctgaggggaccaaaccatcccaacgttaacgttgtcacttgaaaagagaaaggaccaaccggcccccaccccaccccaccccccgcactgctttcgtgaattttgtaaactatgtttgtgtgagtagcgatattgtcagccttctaaaagcaagtggagaacactttaaaatatagaggatttctttttttataagaaaatgctaaatatttatggccatgtacacgttctgacaactggtggcaaatttcgctccttcgttgtaaatatcgctagtgatcgttgccaaatgacgttctggatgggcctgtatccccaccgggcctgcctctctttgtcgtttgttttcggtgggttatgtttgagttgcatcgggtttttatttttatttttatttttattttatttttattttatttttaatttgttcagtgcaaacatttttcaactatgaaaaaggaaaatatgtgtagggaactcaccccccgggccccctttttcgtctcctgagcatcggagcttggaactcagcagtttcacgtggtttccccaagagngccgggc

SEQ ID NO: 19 - protein, 273 aa, Mus musculus
MSLTNTKTGFSVKDILDLPDTNDEDGSVAEGPEEESEGPEPAKRAGPLGQGALDAVQSLPLKSPFYDSSDNPYTRWLASTEGLQYSLHGLAASAPPQDSSSKSPEPSADESPDNDKETQGGGGDAGKKRKRRVLFSKAQTYELERRFRQQRYLSAPEREHLASLIRLTPTQVKIWFQNHRYKMKRARAEKGMEVTPLPSPRRVAVPVLVRDGKPCHALKAQDLAAATFQAGIPFSAYSAQSLQHMQYNAQYSSASTPQYPTAHPLVQAQQWTW

SEQ ID NO: 20 - DNA, 68 bp, Artificial
  Synthetic Construct; siRNA mTm4 ob184_sense
gatccccgccatattgtctctttgtatattcaagagatatacaaagagacaatatggctttttggaaa

SEQ ID NO: 21 - DNA, 68 bp, Artificial
  Synthetic Construct; siRNA mTm4 ob184_antisense
agcttttccaaaaagccatattgtctctttgtatatctcttgaatatacaaagagacaatatggcggg

SEQ ID NO: 22 - DNA, 72 bp, Artificial
  Synthetic Construct; siRNA mTm4 idt7_sense
gatccccgcctgtttgactaatgtgtctggttcaagagaccagacacattagtcaaacaggctttttggaaa

SEQ ID NO: 23 - DNA, 72 bp, Artificial
  Synthetic Construct; siRNA mTm4 idt7_antisense
agcttttccaaaaagcctgtttgactaatgtgtctggtctcttgaaccagacacattagtcaaacaggcggg

SEQ ID NO: 24 - DNA, 70 bp, Artificial
  Synthetic Construct; siRNA mTm4 ob188_sense
gatccccccacgatggtgattatctaaattcaagagagcctgtttgactaatgtgtctggtttttggaaa

SEQ ID NO: 25 - DNA, 70 bp, Artificial
  Synthetic Construct; siRNA mTm4 ob188_antisense
agcttttccaaaaaccacgatggtgattatctaaatctcttgaagcctgtttgactaatgtgtctggggg

SEQ ID NO: 26 - DNA, 64 bp, Artificial
  Synthetic Construct; siRNA mTm4 dh1_sense
gatccccgcataactcaagtggataattcaagagattatccacttgagttatgctttttggaaa

SEQ ID NO: 27 - DNA, 64 bp, Artificial
  Synthetic Construct; siRNA mTm4 dh1_antisense
agcttttccaaaaagcataactcaagtggataatctcttgaattatccacttgagttatgcggg

SEQ ID NO: 28 - DNA, 1354 bp, Mus musculus
agtgtctccaaggttcaaagaactctgatatttggtcccacttttcttattgaccccggtacagccactggaacgtgctgctgcaatccctgaggagaaccgtcaagctggtggcgacgagcctttgatctctgtgctttcctgtgaccccccagcatgtgtactgggggctgtgccaggtgcctggggggcaccctcattcccctggctgtgtttggcctcctggcaaatatcctgttgttctttcctggaggaaaagtggtgaacgacaaaagccacctttcggatgaggtctggtacttcggaggaatattgggaagtggagtcttgatgatcttccctgcgcttgtgttcttgggcctgcagaacaacgactgctgcggatgctgtggcaatgagggctgtgggaagcgatttgcgatgttcacctccacgttgtttgctgtgattggattcttaggtgctggctactcatttatcgtctcagctgtttctatcaacaagggtcctaaatgcttcatggccaatggtacatggggataccccttccacgatggtgattatctaaaagaccaagccttgtggagcgagtgcgaagagccccgtgatgtggtcccctggaacctcaccctcttctccattctgctggtcatcggagggatccagatggttctctgtgccatccaggtgatcaatggcctcctgggaactctctgtggagactgccagtgctgtggctgctgtgggggtgatggaccagtctaacaggttatgatgagctgctccacgtctacagtccagcgcgtgggaagacatgccccaggcccagccctgaccacatgtgttgctaattcctgtctggttcctctctggcagagcttgggaggcacaagagatccttttctctttccaaacagctttgcacaatctagaaattcggtggggttttttttttttttcaaataaaaaactagttagataactttacaaattaggtcctttcttgaattccagaacgagttcaaagtagttttttttttttcattctttcccataagaaaagacaaaatagacaaggtcacttatcctgccatattgtctctttgtatataaagatgttcatattttaggaatatttgcataactcaagtggataaaacattaaaatgaaaaccagtttttcaggatgcataggtaaggaatgattgctatattatatataatttttatgtgaagcctgtttgactaatgtgtctggactacttgtaactagttttatgagcctgtcataatttgtcagggttcccatatgtatattaagttaattaaattcagaattaaaaaaaaaaaaaaa

SEQ ID NO: 29 - protein, 202 aa, Mus musculus
MCTGGCARCLGGTLIPLAVFGLLANILLFFPGGKVVNDKSHLSDEVWYFGGILGSGVLMIFPALVFLGLQNNDCCGCCGNEGCGKRFAMFTSTLFAVIGFLGAGYSFIVSAVSINKGPKCFMANGTWGYPFHDGDYLKDQALWSECEEPRDVVPWNLTLFSILLVIGGIQMVLCAIQVINGLLGTLCGDCQCCGCCGGDGPV

SEQ ID NO: 30 - DNA, 1066 bp, Danio rerio
atgaattcagggtgtgtgtgtgtgagactcagagatcaaactttctgctagggtcttttatcaacactgtagagattacagaggttattctcaggcgtcaggattaaacctatctcacatcatgtgtaccggaaaatgtgcattttgcgtcgggacgagcctgtacccgctggcggtcatctccataatctgtaatattattctgttttttcctggctggggtgttaagtactcgcagaatggacaacttactgaggaggtcaagtacatggggggactggtcggagggggagtaatggtgctgattccagcatttcacattcacctgactggaaaacaaggttgctgtgctaatcgctgtgggatgttcctgtccattttatttgcagcagtaggcgttgttggcgctctctatagtttcatagtagcgctgatgggtctaattaatgggccatactgtttatctagctttagttggaccactccctttaaagacaggaacgagagttaccttaaggaccgtgactcatggaaagattgcaccgagccgaagaatgtggtggagtttaatgtgggtctgttctccacactactggtgaccagtgctgtgcagctcgtcctttgtgcagtgcagatgatcaacggtcttttcggctgcctctgtggaacctgcaaaaaagacaaggggccgctgtaactccttactgctccaaacgcccgggagaccatatccatcacacctgccacccagcctcccaatcagctaccgagcgccatcttctggaagatgttgaagaatttaatggaatgtgtttgtactttgtgtgtgtacacgtaaatcttttcatattttgatgtttattgcttacattttttcttactactgttttttaagtaaaggcatatgcgtaatgacttctagagccaatcatattttttaaagaaagttgcgttaattaatggcataacactcgttttgtatgcatctgttttttataggttaaagattaaatgaacatactaagaataaaatccaaactgttacacaaaaaaaaaaaaaaaaaaa

SEQ ID NO: 31 - protein, 191 aa, Danio rerio
MCTGKCAFCVGTSLYPLAVISIICNIILFFPGWDVKYSQNGQLTEEVKYMGGLVGGGVMVLIPAFHIHLTGKQGCCANRCGMFLSILFAAVGVVGALYSFIVALMGLINGPYCLSSFSWTTPFKDRNESYLKDRDSWKDCTEPKNVVEFNVGLFSTLLVTSAVQLVLCAVQMINGLFGCLCGTCKKDKGPL

SEQ ID NO: 32 - DNA, 2092 bp, Homo sapiens
gcggccgccggagcccgagctgacgccgccttggcacccctcctggagttagaaactaaggccggggcccgcggcgctcggcgcgcaggccgcccggcttcctgcgtccatttccgcgtgctttcaaagaagacagagagaggcactgggttgggcttcatttttttcctccccatccccagtttctttctctttttaaaaataataattatcccaataattaaagccaattcccccctcccctcccccagtccctccccccaactcccccctcccccgcccgccggggcaggggagcgccacgaattgaccaagtgaagctacaactttgcgacataaattttggggtctcgaaccatgtcgctgaccaacacaaagacggggttttcggtcaaggacatcttagacctgccggacaccaacgatgaggagggctctgtggccgaaggtccggaggaagagaacgaggggcccgagccagccaagagggccgggccgctggggcagggcgccctggacgcggtgcagagcctgcccctgaagaaccccttctacgacagcagcgacaacccgtacacgcgctggctggccagcaccgagggccttcagtactccctgcacggtctggctgccggggcgccccctcaggactcaagctccaagtccccggagccctcggccgacgagtcaccggacaatgacaaggagaccccgggcggcgggggggacgccggcaagaagcgaaagcggcgagtgcttttctccaaggcgcagacctacgagctggagcggcgctttcggcagcagcggtacctgtcggcgcccgagcgcgaacacctggccagcctcatccgcctcacgcccacgcaggtcaagatctggttccagaaccaccgctacaagatgaagcgcgcccgggccgagaaaggtatggaggtgacgcccctgccctcgccgcgccgggtggccgtgcccgtcttggtcagggacggcaaaccatgtcacgcgctcaaagcccaggacctggcagccgccaccttccaggcgggcattcccttttctgcctacagcgcgcagtcgctgcagcacatgcagtacaacgcccagtacagctcggccagcaccccccagtacccgacagcacaccccctggtccaggcccagcagtggacttggtgagcgccgccccaacgagactcgcggccccaggcccaggccccaccccggcggcggtggcggcgaggaggcctcggtccttatggtggttattattattattataattattattatggagtcgagttgactctcggctccactagggaggcgccgggaggttgcctgcgtctccttggagtggcagattccacccacccagctctgcccatgcctctccttctgaaccttgggagagggctgaactctacgccgtgtttacagaatgtttgcgcagcttcgcttctttgcctctccccggggggaccaaaccgtcccagcgttaatgtcgtcacttgaaaacgagaaaaagaccgaccccccacccctgctttcgtgcattttgtaaaatatgtttgtgtgagtagcgatattgtcagccgtcttctaaagcaagtggagaacactttaaaaatacagagaatttcttcctttttttaaaaaaaaataagaaaatgctaaatatttatggccatgtaaacgttctgacaactggtggcagatttcgcttttcgttgtaaatatcggtggtgattgttgccaaaatgaccttcaggaccggcctgtttcccgtctgggtccaactcctttctttgtggcttgtttgggtttgttttttgttttgtttttgtttttgcgttttcccctgctttcttcctttctctttttattttattgtgcaaacatttctcaaatatggaaaagaaaaccctgtaggcagggagccctctgccctgtcctccgggccttcagccccgaacttggagctcagctattcggcgcggttccccaacagcgccgggcgcagaaagctttcgattttttaaataagaattttaataaaaatcctgtgtttaaaaaagaaaaaaagaaaaaa

SEQ ID NO: 33 - protein, 272 aa, Homo sapiens
MSLTNTKTGFSVKDILDLPDTNDEEGSVAEGPEEENEGPEPAKRAGPLGQGALDAVQSLPLKNPFYDSSDNPYTRWLASTEGLQYSLHGLAAGAPPQDSSKSPEPSADESPDNDKETPGGGGDAGKKRKRRVLFSKAQTYELERRFRQQRYLSAPEREHLASLIRLTPTQVKIWFQNHRYKMKRARAEKGMEVTPLPSPRRVAVPVLVRDGKPCHALKAQDLAAATFQAGIPFSAYSAQSLQHMQYNAQYSSASTPQYPTAHPLVQAQQWTW

SEQ ID NO: 34 - protein, 16 aa, Artificial
  Synthetic construct; antibody epitope
CGTWGYPFHDGDYLKD

SEQ ID NO: 35 - DNA, 82 bp, Artificial
  Synthetic Construct
agtcacttttttttcaagaaatcttttataagaattaaacccatgcttaatattaataactaggcggccgcatttaaatggc

SEQ ID NO: 36 - DNA, 83 bp, Artificial
  Synthetic Construct
ctacaggcagatccagaatatctagctctttatcccttacaggggcagccagattgaagaacatcgatgatatcagatctgcc

SEQ ID NO: 37 - DNA, 86 bp, Artificial
  Synthetic Construct
aagctggtggcgacgagcctttgatctctgtgctttcctgtgaccccccagcatgtgtgctcgagacgtagaaagccagtccgcag

SEQ ID NO: 38 - DNA, 90 bp, Artificial
  Synthetic Construct
caagactccacttcccaatattcctccgaagtaccagacctcatccgaaaggtggcttttcgtacgcaaaattcagaagaactcgtcaag

SEQ ID NO: 39 - DNA, 21 bp, Artificial
  Synthetic Construct
catcagggtgtcatggttggt

SEQ ID NO: 40 - DNA, 22 bp, Artificial
  Synthetic Construct
tctcttgctctgagcctcatca

SEQ ID NO: 41 - DNA, 19 bp, Artificial
  Synthetic Construct
tgggacagaaagacagcta

SEQ ID NO: 42 - DNA, 22 bp, Artificial
  Synthetic Construct
tcctcagtccgactcagaaacc

SEQ ID NO: 43 - DNA, 22 bp, Artificial
  Synthetic Construct
gcttctcttctgcccactcttg

SEQ ID NO: 44 - DNA, 14 bp, Artificial
  Synthetic Construct
agggtcgaaggcca

SEQ ID NO: 45 - DNA, 20 bp, Artificial
  Synthetic Construct
aagcgaggagacgatccaaa

SEQ ID NO: 46 - DNA, 21 bp, Artificial
  Synthetic Construct
tccaacacacaccagcaaatg

SEQ ID NO: 47 - DNA, 21 bp, Artificial
  Synthetic Construct
acatttcatatcatctcatcc

SEQ ID NO: 48 - DNA, 19 bp, Artificial
  Synthetic Construct
gagccccttctgggtttcc

SEQ ID NO: 49 - DNA, 22 bp, Artificial
  Synthetic Construct
aagtcagccacctcagtttcct

SEQ ID NO: 50 - DNA, 16 bp, Artificial
  Synthetic Construct
tcctcctaaatctgcc

SEQ ID NO: 51 - DNA, 20 bp, Artificial
  Synthetic Construct
aaccacggacagcatccaat

SEQ ID NO: 52 - DNA, 20 bp, Artificial
  Synthetic Construct
tttgcggacgtgtcttgaga

SEQ ID NO: 53 - DNA, 23 bp, Artificial
  Synthetic Construct
tcattacacggcctgtccgcgaa

SEQ ID NO: 54 - DNA, 18 bp, Artificial
  Synthetic Construct
cacacgcacgcatggaaa

SEQ ID NO: 55 - DNA, 19 bp, Artificial
  Synthetic Construct
tcctcggcctcgaccatat

SEQ ID NO: 56 - DNA, 15 bp, Artificial
  Synthetic Construct
cagtggacaggccct

SEQ ID NO: 57 - DNA, 19 bp, Artificial
  Synthetic Construct
gccaaactccgccaacttc

SEQ ID NO: 58 - DNA, 20 bp, Artificial
  Synthetic Construct
ctggcgagttcctgttttcc

SEQ ID NO: 59 - DNA, 17 bp, Artificial
  Synthetic Construct
atctctcctcagccctg

SEQ ID NO: 60 - DNA, 22 bp, Artificial
  Synthetic Construct
gcaaagttctagagggcatgga

SEQ ID NO: 61 - DNA, 19 bp, Artificial
  Synthetic Construct
cccggctgtctgtcttggt

SEQ ID NO: 62 - DNA, 17 bp, Artificial
  Synthetic Construct
tggtacggaaggtggag

SEQ ID NO: 63 - DNA, 3121 bp, Artificial
  Synthetic Construct
ttaattaagaattcnnnnnnccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgacnnnnnntctagggctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttctagacattaaataacttcgtataatgtatgctatacgaagttatgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgactgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttataacttcgtataatgtatgctatacgaagttatttaattaa

SEQ ID NO: 64 - DNA, 1212 bp, Artificial
  Synthetic Construct
ccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgacnnnnnntctagggctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagct

SEQ ID NO: 65 - DNA, 34 bp, Artificial
  Synthetic Construct
ataacttcgtataatgtatgctatacgaagttat

SEQ ID NO: 66 - DNA, 1801 bp, Artificial
  Synthetic Construct
gaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgactgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctt